SEQUENCE LISTING (56 sequences)

SEQ ID NO: 1 - DNA, 20 bp, synthetic construct
  source 1..20: mol_type = other DNA
ccggagaggtgacttcacag

SEQ ID NO: 2 - DNA, 20 bp, synthetic construct
  source 1..20: mol_type = other DNA
ttctgcaagtgcatcatcgt

SEQ ID NO: 3 - DNA, 20 bp, synthetic construct
  source 1..20: mol_type = other DNA
cccactcacctgctgctact

SEQ ID NO: 4 - DNA, 20 bp, synthetic construct
  source 1..20: mol_type = other DNA
tctggacccattccttcttg

SEQ ID NO: 5 - DNA, 23 bp, synthetic construct
  source 1..23: mol_type = other DNA
tctcagattcacaactgttcgtg

SEQ ID NO: 6 - DNA, 23 bp, synthetic construct
  source 1..23: mol_type = other DNA
agaaaatgaggtcggtctcacta

SEQ ID NO: 7 - DNA, 23 bp, synthetic construct
  source 1..23: mol_type = other DNA
tggccatctacaagaagtcacag

SEQ ID NO: 8 - DNA, 18 bp, synthetic construct
  source 1..18: mol_type = other DNA
atcggagcagcgctcatg

SEQ ID NO: 9 - DNA, 20 bp, synthetic construct
  source 1..20: mol_type = other DNA
gttccgcacaggagcaaagt

SEQ ID NO: 10 - DNA, 17 bp, synthetic construct
  source 1..17: mol_type = other DNA
acggcgcaactgctcac

SEQ ID NO: 11 - DNA, 18 bp, synthetic construct
  source 1..18: mol_type = other DNA
agtaccccattgaacacg

SEQ ID NO: 12 - DNA, 18 bp, synthetic construct
  source 1..18: mol_type = other DNA
aatgccagtggtacgacc

SEQ ID NO: 13 - DNA, 80 bp, Homo sapiens
  source 1..80: mol_type = genomic DNA
aggacccttccagagggccccccctcaatcctgttgtgcctaattcagagggttgggtggaggctctcctgaagggctct

SEQ ID NO: 14 - RNA, 21 nt, Homo sapiens
  source 1..21: mol_type = other RNA
agggccccccctcaatcctgt

SEQ ID NO: 15 - DNA, 84 bp, Homo sapiens
  source 1..84: mol_type = genomic DNA
acgtcagggaaaggattctgctgtcggtcccactccaaagttcacagaatgggtggtgggcacagaatctggactctgcttgtg

SEQ ID NO: 16 - RNA, 25 nt, Homo sapiens
  source 1..25: mol_type = other RNA
aaaggattctgctgtcggtcccact

SEQ ID NO: 17 - RNA, 22 nt, synthetic construct
  source 1..22: mol_type = other RNA
tggtgggcacagaatctggact

SEQ ID NO: 18 - RNA, 22 nt, synthetic construct
  source 1..22: mol_type = other RNA
tccagattctgtgcccaccatt

SEQ ID NO: 19-54 - skipped (36 entries)

SEQ ID NO: 55 - protein, 11 aa, synthetic construct
ACSSSPSKHCG

SEQ ID NO: 56 - protein, 11 aa, synthetic construct
ACTGSTQHQCG

(19) United States
(12) Patent Application Publication
(10) Pub. No.: US 2026/0130937 A1
(43) Pub. Date: May 14, 2026

(54) COMPOSITION AND METHODS TO REDUCE INFLAMMATION AND PROMOTE REJUVENATION

(71) Applicant: Augusta University Research Institute, Inc., Augusta, GA (US)

(72) Inventor: Raghavan Pillai Raju, Martinez, GA (US)

(21) Appl. No.: 19/312,420

(22) Filed: Aug. 28, 2025

Related U.S. Application Data
(60) Provisional application No. 63/689,441, filed on Aug. 30, 2024.

(51) Int. Cl. A61K 35/16; A61K 9/50; A61P 29/00; A61P 31/00; C12N 15/113

(52) U.S. Cl. CPC A61K 35/16; A61K 9/5068; A61P 29/00; A61P 31/00; C12N 15/1136; C12N 15/1137; C12Y 114/13039; C12N 2310/141

(57) ABSTRACT

Compositions containing include extracellular vesicles or MicroRNA isolated therefrom) derived from a sample such as plasma, obtained from a young donor subject (hereinafter, Young EVs, or Y-EVs), are provided. The Y-EVs are preferably cell-free compositions. The mircorRNA isolated from Y-EVs include miRNA-296-5p and miR-541-5p (referred to herein as “Young” miRNA-296-5p and miR-541-5p or Y-mirRNA-296-5p and Y-miR-541-5p.

The Y-EVs, Y-mirRNA-296-5p or functionally active variants thereof, and/or Y-mirRNA-296-5p or functionally active variants thereof, or a combination thereof, are included in suitable pharmaceutical formulations which include an effective amount of the EVs to, for example, serve a cosmeceutical and therapeutic application such as improving skin, treating an inflammatory skin-related disease or disorder, or enhancing recovering from sepsis.

### CROSS-REFERENCE TO RELATED APPLICATIONS

This application claims the benefit of and priority to U.S. Provisional Application No. 63/689,441, filed Aug. 30, 2024, which is hereby incorporated herein by reference in its entirety.

### STATEMENT REGARDING FEDERALLY SPONSORED RESEARCH

This invention was made with government support under Grant No. R01AG073338 awarded by National Institutes of Health. The government has certain rights in the invention.

### REFERENCE TO THE SEQUENCE LISTING

The Sequence Listing submitted as an XML file named “AURI_2024-024_US_ST26”, created Jan. 26, 2026, and having a size of 25,950 bytes is hereby incorporated by reference pursuant to 37 C.F.R. § 1.834 (c)(1).

### FIELD OF THE INVENTION

The disclosed invention is generally related to compositions and methods for treating inflammation and sepsis.

### BACKGROUND OF THE INVENTION

Aging is an inevitable biological process and is associated with a progressive decline in physiological function. Though the discovery of methods to extend longevity is much sought after, finding methods for healthy aging may be a more attainable goal. Several agents that modulate autophagy, mitochondrial function, senescence, and antioxidant responses have been shown to promote healthy aging, and their effects may be attributed to the functionally interrelated nature of these biological processes and functions¹⁻³. There have also been several attempts to identify rejuvenating endogenous factors; these include metabolic regulators and various growth factors native to the being⁴.

The resilience of the young organism to injury and infection is well documented⁵⁻⁷. Further, heterochronic parabiosis experiments have demonstrated that young blood can rejuvenate aging systems⁸. However, the factors in the young blood that are responsible for the rejuvenating effect, or that in the old animal that triggers organismal aging, remains unknown or, at best, speculative⁹.

Sepsis is a life-threatening inflammatory disease in response to infection with an age-associated increase in mortality^{18,19}. While patients older than 65 years (elderly) accounted for only 12% of the US population, 64.9% of the sepsis cases were in this age group, with similar data reported from other countries¹⁸. There is still a need for compositions and methods to treat sepsis.

### BRIEF SUMMARY OF THE INVENTION

Compositions containing extracellular vesicles (or microRNA isolated therefrom) derived from a sample such as plasma, obtained from a young donor subject (hereinafter, Young EVs, or Y-EVs), are provided. The Y-EVs are preferably cell-free compositions. In some forms, the mircroRNA isolated from Y-EVs include miRNA-296-5p and miR-541-5p (referred to herein as “Young miRNA-296-5p” and “Young miR-541-5p” or “Y-mirRNA-296-5p” and “Y-miR-541-5p”).

The disclosed compositions include Y-EVs, Y-mirRNA-296-5p or functionally active variants thereof, and/or Y-mirRNA-541-5p or functionally active variants thereof. Thus, the disclosed compositions can include the disclosed miRNA, their mimics, their agomirs, miRNA precursors and miRNA-expressing plasmids.

In some forms, the compositions include a Y-mirRNA-296-5p mimic. In some forms, the compositions include a Y-mirRNA-296-5p agomir.

In some forms, the compositions include a Y-miR-541-5p mimic. In some forms, the compositions include a Y-miR-541-5p agomir.

In some forms, the Y-mirRNA-296-5p and Y-mirRNA-296-5p are obtained from a human plasma sample.

In some forms, the compositions includes hsa-mir-296-5p mature miRNA (5′-AGGGCCCCCCCUCAAUCCUGU-3′); mIRbase Accession ID: MIMAT0000690; SEQ ID NO: 14), or a functional variant thereof for example, a sequence having at least 70, 75, 80, 85, 90, 95 and up to 99% sequence identity to SEQ ID NO:14.

In some forms, the compositions include hsa-miR-541-5p mature miRNA, (AAAGGAUUCUGCUGUCGGUCCCACU (SEQ ID NO: 16); mIRbase Accession ID: MIMAT0004919), or a functional variant thereof, for example, a sequence having at least at least 70, 75, 80, 85, 90, 95 and up to 99% sequence identity to SEQ ID NO:16.

The Y-EVs, Y-mirRNA-296-5p or functionally active variants thereof, and/or Y-mirRNA-296-5p or functionally active variants thereof, or a combination thereof, are independently, or in combination, included in suitable pharmaceutical formulations in an effective amount to, for example, serve as a cosmeceutical and in therapeutic applications such as improving skin, treating an inflammatory skin-related disease or disorder, or enhancing recovering from sepsis. In some forms, the compositions are effective to reduce intracellular pro-inflammatory cytokines such as IL (interleukin)-1β, TNF-α (tumor necrosis factor alpha) and IL-6 and/or inos (inducible nitric oxide synthase).

The disclosed compositions are administered to a subject in need thereof, in an effective amount to reduce one or more symptoms associated with an inflammatory condition or sepsis.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIG. 1A-1B. Effect of age on survival after CLP-induced sepsis. (FIG. 1A) 10-day survival of mice, three different age groups, following CLP-induced sepsis, represented by Kaplan-Meier survival curves show significantly reduced mortality rate in young mice (5-6 weeks) compared to mature (14-16 weeks) or aged (23-26 months) mice. Young vs. mature, p=0.0348; mature vs aged, p=0.0025; young vs. aged, p=0.0002. (FIG. 1A) Body weight data *p<0.05 compared to mature group (n=10/group).

FIG. 2A-2G. Characterization of plasma EVs. (FIG. 2A-2B) Representative elution profile with protein concentration, particle number and particle diameter of EVs isolated from mouse plasma. The EVs were isolated using size exclusion chromatography as described in Methods. Eluted fractions 9-14 were pooled and used. (FIG. 2C-2D) Age-associated change in EV particle size and number. (FIG. 2E) Western blot analysis of exosome markers in EVs isolated from four different age groups. (FIG. 2F) Representative immuno-transmission electron microscopy images demonstrating CD9 and CD63 surface markers on the EVs (FIG. 2G) Cell-free mitochondrial DNA (cf-mtDNA) levels in the plasma EVs change with age. 10⁷ particles per sample were used to isolate the cf-mtDNA (see methods for details). Representative of two independent experiments. n=5/group. Statistical significance was defined as ****=p<0.0001; ***=p<0.001; **=p<0.01; *=p<0.05 for data in respective panels.

FIG. 3A-3D. Plasma EVs from young mice reduce mortality following CLP-induced sepsis. (FIG. 3A) Plasma EVs isolated from young, mature or aged mice were administered (10⁸ particles/dose) to mature mice subjected to CLP or sham surgery at 2, 24 and 48 hours after the surgery. 10-day survival following CLP-induced sepsis in each group is represented by Kaplan-Meier curves, and show most significant survival in mice that received young EVs. (FIG. 3B, 3C) Sepsis score in different groups after EVs treatment following sepsis on day 1 (FIG. 3B) and day 2 (FIG. 3C). The data is a composite of three independent experiments. Veh. (n=18), Young (n=26), Mature (n=26) and Aged (n=18). Statistical significance was defined as p<0.05. ****=p<0.0001; *=p<0.001; **=p<0.01; *=p<0.05. (FIG. 3D) SPECT/CT imaging shows EVs administered through tail vein homing to multiple internal organs (Representative figure).

FIG. 4A-4C. Age-dependent effect of plasma EVs on inflammation and senescence. (FIG. 4A) Plasma EVs isolated from mice of three different ages were incubated with MEFs challenged with LPS for 24 h and the percentage of total LDH released in the supernatant was measured. Each age group was assayed in six replicates. Young and mature EVs significantly reduced the cell death. (FIG. 4B-4D) LPS-treated MEF cells were cultured with EVs and the expression of inflammatory markers measured after 24 hours by real time PCR. Fold induction for each gene was normalized to vehicle control. (FIG. 4E-4G) Flow cytometry analysis of the expression of p16 and p21 in MEF cells. Doxorubicin-treated cells were co-cultured with EVs for 24 hours and tested for the expression of p16 and p21 by flow cytometry. Results shown as Mean±SEM (n=4-6). Representative of two independent experiments. Statistical significance (One-way Anova) is expressed as * p<0.05; ** p<0.01; *** p<0.001; **** p<0.0001.

FIG. 5A-5C: Age-associated changes in miRNAs in the cargo of plasma EVs. (FIG. 5A) Left: Sample correlation and cluster analysis of differentially expressed miRNAs with age in plasma EVs (Y=Young, M=Mature). Right: Heatmap of 32 differentially expressed miRNAs which are significant for at least one age against the young group. The two miRNAs (miRNA296-5p and miRNA541-5p) that showed progressive decline in their levels with age are shown by arrows (FIG. 5B) Graphical representation of miRNA expression data as obtained from miRNA sequencing (FIG. 5C) Validation of miRNA296-5p and miRNA541-5p levels in plasma EVs from each of the age group. miR-296-5p and miR541-5p expression level in mouse liver, lung, kidney and heart demonstrate significant reduction with age. Data expressed as mean±SEM; Statistical significance (one-way Anova) was defined as ****=p<0.01; ***=p<0.01; **=p<0.01; *=p<0.05.

FIG. 6A-6B. miR296 and miR541 reduce LPS induced inflammation. (FIG. 6A) MEF cells were transfected with miR296-5p mimic or miRNA541-5p mimic or negative control mRNA and incubated in the absence or presence of LPS. Cytokine expression was measured by real time PCR. Mean values are shown as fold induction relative to β-actin and normalized to miRNA negative control. (FIG. 6B) MEF cells were transfected with miRNA296-5p inhibitor or miRNA541-5p inhibitor (antagomirs) and cytokine expression was measured by RT-qPCR. Mean values are shown as fold induction relative to β-actin and normalized to miRNA negative control. Data expressed as mean±SEM; Statistical significance was defined as **=p<0.01: *=p<0.05. n=4. Representative of two independent experiments.

FIG. 7A-7F: Young EVs and the miRNAs miR296-5p and 541-5p improve wound healing in an in vitro scratch assay model. (FIG. 7A) Rat PMVECs were plated, scratched and incubated with EVs isolated from young and aged mice at 0-hour, 24 hour and 48-hour intervals. (FIG. 7B) Comparison of the area of scratch filled (migration) cells after treatment with young or aged EV at different time points. (FIG. 7C) Rat PMVECs transfected with mimics of miR296-5p or miR541-5p, were plated and scratched. Observed after 0 hour, 24 hour and 48-hour intervals. (FIG. 7D) The area of scratch filled in cells at different time points after treatment with miR296-5p and miR541-5p mimics. (FIG. 7E) Rat PMVECs incubated with young EVs transfected with miR-inhibitor-296 and miR-inhibitor-541, scratched and observed at 0 hour, 24 hour and 48-hour intervals. (FIG. 7F) The change in the area of scratch after treatment with EVs transfected with antagomirs of miR296-5p and miR541-5p at different time points. (FIG. 7G) Panel shows EVs isolated from the plasma of young mice were transfected with a labelled transfection control (red) using lipofectamine and incubated with PMVECs. Nucleus is stained with Hoechst. Images were capture in the Echo Revolve microscope (Life technologies) at 200× magnification.

FIG. 8A-8B: miR296 improves survival following CLP-induced sepsis. (FIG. 8A) Kaplan-Meier survival curves of mice subjected to CLP-induced sepsis and treated (i.p.) with miR-296-5p negative control (n=15) or miR-296-5p mimic (n=15). Composite data from three independent experiments. Veh. vs. miR-296-5p. p=0.046. (FIG. 8B) Twenty-four hours after CLP-surgery, sepsis score showed less severity in the group with mi296 administration.

### DETAILED DESCRIPTION OF THE INVENTION

The disclosed method and compositions can be understood more readily by reference to the following detailed description of particular embodiments and the Example included therein and to the Figures and their previous and following description.

The present studies demonstrate the resilience of the young animal, at or before puberty, to polymicrobial sepsis and show an age-dependent effect of plasma extracellular vesicles on the outcome following sepsis. The EVs (extracellular vesicles) from the young animals were cytoprotective, anti-inflammatory, and reduced cellular senescence markers. MicroRNA sequencing of the extracellular vesicles showed an age-associated signature and identified two miRNAs, miR296-5p and miR-541-5p, to be progressively reducing in their levels in the blood plasma with increasing age. These miRNA levels decline with age in multiple organs, and they are anti-inflammatory. When administered intraperitoneally, miR-296-5p reduced mortality in the mouse model of sepsis.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### I. Definitions

As used herein, the term “carrier” or “excipient” refers to an organic or inorganic ingredient, natural or synthetic inactive ingredient in a formulation, with which one or more active ingredients are combined.

As used herein, the term “gene” refers to a nucleic acid (e.g., DNA or RNA) sequence that including coding sequences necessary for the production of a polypeptide. RNA (e.g., including, but not limited to, mRNA, tRNA and rRNA) or precursor. The polypeptide, RNA, or precursor can be encoded by a full-length coding sequence or by any portion thereof. The term also encompasses the coding region of a structural gene and the sequences located adjacent to the coding region on both 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation.

As used herein, “mammal” includes both humans and non-humans and include but is not limited to humans, non-human primates, canines, felines, murines, bovines, equines, and porcines.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “pharmaceutically acceptable” means a non-toxic material that does not interfere with the effectiveness of the biological activity of the active ingredients.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “inhibit” or “reduce” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” or “reduce” can also mean to hinder or restrain the synthesis, expression or function of a protein relative to a standard or control. This can include, but is not limited to, the complete ablation of the activity, response, condition, or disease. Inhibition may also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels.

As used herein, “senescence” refers to the point at which a cell is no longer capable of undergoing mitosis (cell division).

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and Care disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the subgroup of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### II. Compositions

The disclosed compositions can in some forms include isolated YEVs and/or isolated miR296-5p and/or isolated miR541-5p (each referred to herein as “compound(s) or active compound(s)”), and can be formulated into a pharmaceutical composition using a suitable carrier to administration to a subject in need thereof.

### 1. Extracellular Vesicles (EVs)

Extracellular vesicles are lipid bilayer-delimited particles that are naturally released from a cell and, unlike a cell, cannot replicate. EVs range in diameter from near the size of the smallest physically possible unilamellar liposome (around 20-30 nanometers) to as large as 10 microns or more, although the vast majority of EVs are smaller than 200 nm.

Diverse EV subtypes have been proposed including ectosomes, microvesicles (MV), microparticles, exosomes, oncosomes, apoptotic bodies (AB), tunneling nanotubes (TNT), and more (Yáñez-Mó, et al., J Extracell Vesicles, 4:27066 (2015) doi:10.3402/jev.v4.27066. PMC 4433489). These EV subtypes have been defined by various, often overlapping, definitions, based mostly on biogenesis (cell pathway, cell or tissue identity, condition of origin) (Théry, et al., J Extracell Vesicles, 7 (1): 1535750 (2018). doi:10.1080/20013078.2018.1535750, which is specifically incorporated by reference herein in its entirety). However, EV subtypes may also be defined by size, constituent molecules, function, or method of separation. As discussed in Théry, et al., subtypes of EVs may be defined by:
a) physical characteristics of EVs, such as size (“small EVs” (sEVs) and “medium/large EVs” (m/IEVs), with ranges defined, for instance, respectively, <100 nm or <200 nm [small], or >200 nm [large and/or medium]) or density (low, middle, high, with each range defined);
b) biochemical composition (CD63+/CD81+-EVs, Annexin A5-stained EVs, etc.); or
c) descriptions of conditions or cell of origin (podocyte EVs, hypoxic EVs, large oncosomes, apoptotic bodies).

Thus, in some embodiments, the composition is or includes one or more EV subtypes defined according (a), (b), or (c) as discussed above.

In some embodiments, the vesicles are or include exosomes, which may also be referred as, or include, “small EVs”, “sEVs”, etc. Exosomes possess the surface proteins that promote endocytosis and they have the potential to deliver macromolecules. Also, if the exosomes are obtained from the same individual as they are delivered to, the exosomes will be immunotolerant.

Exosomes are vesicles with the size of 30-150 nm, often 40-100 nm, and are observed in most cell types. Exosomes are often similar to MVs with an important difference: instead of originating directly from the plasma membrane, they are generated by inward budding into multivesicular bodies (MVBs). The formation of exosomes includes three different stages: (1) the formation of endocytic vesicles from plasma membrane, (2) the inward budding of the endosomal vesicle membrane resulting in MVBs that consist of intraluminal vesicles (ILVs), and (3) the fusion of these MVBs with the plasma membrane, which releases the vesicular contents, known as exosomes.

Exosomes have a lipid bilayer with an average thickness of ˜5 nm (see e.g., Li, Theranostics, 7 (3): 789-804 (2017) doi: 10.7150/thno.18133). The lipid components of exosomes include ceramide (sometimes used to differentiate exosomes from lysosomes), cholesterol, sphingolipids, and phosphoglycerides with long and saturated fatty-acyl chains. The outer surface of exosomes is typically rich in saccharide chains, such as mannose, polylactosamine, alpha-2,6 sialic acid, and N-linked glycans.

Many exosomes contain proteins such as platelet derived growth factor receptor, lactadherin, transmembrane proteins and lysosome associated membrane protein-2B, membrane transport and fusion proteins like annexins, flotillins, GTPases, heat shock proteins, tetraspanins, proteins involved in multivesicular body biogenesis, as well as lipid-related proteins and phospholipases. These characteristic proteins therefore serve as good biomarkers for the isolation and quantification of exosomes. Another key cargo that exosomes can carry is nucleic acids including deoxynucleic acids (DNA), coding and non-coding ribonucleic acid (RNA) like messenger RNA (mRNA) and microRNA (miRNA).

In some embodiments, the vesicles include or are one or more alternative extracellular vesicles, such as ABs, MVs, TNTs, or others discussed herein or elsewhere.

ABs are heterogenous in size and originate from the plasma membrane. They can be released from all cell types and are about 1-5 μm in size.

MVs with the size of 20 nm-1 μm are formed due to blebbing with incorporation of cytosolic proteins. In contrast to ABs, the shape of MVs is homogenous. They originate from the plasma membrane and are observed in most cell types.

TNTs are thin (e.g., 50-700 nm) and up to 100 μm long actin containing tubes formed from the plasma membrane.

In some embodiments, the EVs are between about 20 nm and about 500 nm. In some embodiments, the EVs are between about 20 nm and about 250 nm or 200 nm or 150 nm or 100 nm.

i. EV miroRNA

The disclosed compositions can in some forms include isolated miR296-5p and/or miR541-5p obtained from a young mammalian donor such as a young human.

MicroRNAs (miRNAs) are small non-coding 21-23 nt RNAs, which are essential in cell proliferation, differentiation, apoptosis, chemoresistance, and stress response. MiRNA biogenesis begins in the nucleus with transcription, followed by multiple processing steps to produce mature miRNAs. The biogenesis of mature miRNAs starts with RNA polymerase II processing of long non-protein coding RNA primary transcripts, called primary miRNAs. These transcripts are further processed by DROSHA and its binding partners, such as DGCR8, leading to precursor miRNAs (pre-miRNAs). Pre-miRNAs are translocated into the cytoplasm via exportin 5 and then bind to DICER and RNA-induced silencing complex (RISC), which includes argonaute proteins. The mature miRNAs associate with an RISC and become active miRNAs that suppress the transcription of target genes, inducing mRNA degradation or the inhibition of protein translation.

MiR-296 is located in chromosomal region 20q13.32; it has a high degree of sequence conservation among species and plays important roles in many biological processes. Mature miR-296 is called miR-296-5p and miR-296-3p if derived from the 5′ arm and 3′ arm of precursor miR-296, respectively. miR-296-3p and miR-296-5p are two transcripts from the same gene; the latter is often simply referred to as miR-296 (Zhu, et al., Research and Practice, 214 (12):1915-1922 (2018)). In preferred forms, the miR-296-3p and miR-541-5p are each a human miR-296-3p and hsa-miR-541-5p.

In some forms, the compositions include a functional variant/mimic of miR296-5p and/or miR541-5p. Thus, the disclosed compositions can include the disclosed miRNA, miRNA mimics, miRNA agomirs, miRNA precursors and miRNA-expressing plasmids. miRNA mimics are synthetic double-stranded miRNA-like RNA molecules that can simulate endogenous miRNAs. miRNA Mimics are chemically modified, double-stranded miRNA-like RNA which are designed to copy the functionality of mature endogenous miRNA upon transfection. miRNA agomirs are artificial double-stranded miRNA mimics with more chemical modifications. Compared with miRNA mimics, these chemical modifications enhance the stability and activity of miRNA agomirs.

Human miR296: NCBI Gene ID: 407022; NCBI Reference Sequence: NC_000020.11 (Homo Sapiens Chromosome 20, GRCh38.p14 (nucleotides 58817615 to 58817694) is shown below:

(SEQ ID NO: 13)
AGGACCCTTCCAGAGGGCCCCCCCTCAATCCTGTTGTGCCT
AATTCAGAGGGTTGGGTGGAGGCTCTCCTGAAGGGCTCT

In some forms, the compositions includes hsa-mir-296-5p (5′-AGGGCCCCCCCUCAAUCCUGU-3′ (Mature hsa-miR-296-5p (mIRbase Accession ID: MIMAT0000690; SEQ ID NO:14), or a functional variant thereof for example, a sequence having at least 70, 75, 80, 85, 90, 95 and up to 99% sequence identity to SEQ ID NO:14.

Human miR 541: NCBI Gene ID: 100126308; NCBI Reference Sequence: NC_000014.9 (Homo sapiens chromosome 14, GRCh38.p14 Primary Assembly (nucleotides 101064495-101064578), is shown below:

(SEQ ID NO: 15)
ACGTCAGGGAAAGGATTCTGCTGTCGGTCCCACTCCAAAGTT
CACAGAATGGGTGGTGGGCACAGAATCTGGACTCTGCTTGTG

In some forms, the compositions includes hsa-miR-541-5p mature miRNA, AAAGGAUUCUGCUGUCGGUCCCACU (SEQ ID NO: 16); mIRbase Accession ID: MIMAT0004919), or a functional variant thereof, for example, a sequence having at least at least 70, 75, 80, 85, 90, 95 and up to 99% sequence identity to SEQ ID NO:16. Xu, et al., (https://doi.org/10.1002/cam4.1491) discloses exemplary sequences of miR-541-mimics as follows: sense strand: 5′-UGGUGGGCACAGAAUCUGGACU-3′ (SEQ ID NO: 17); and antisense strand: 5′-UCCAGAUUCUGUGCCCACCAUU-3′ (SEQ ID NO: 18).
An hsa-miR-541-5p agomir is available from Mechem Express, Cat. No.: HY-R01623Aca; An hsa-miR-541-5p mimic is available from Mechem Express, Cat. No.: HY-R01623

As used herein, the term “percent (%) sequence identity” is defined as the percentage of nucleotides in a candidate sequence that are identical with the nucleotides in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotide sequence C to, with, or against a given nucleic acid sequence D (which can alternatively be phrased as a given sequence C that has or includes a certain % sequence identity to, with, or against a given sequence D) is calculated as follows:

100 times the fraction W/Z,
where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

Usually, naked RNA is highly susceptible to degradation by abundant ribonucleases in the blood and to phagocytosis by the reticuloendothelial system (RES). Chemical modifications can increase the stability of oligonucleotides for in vivo delivery. Chemical modifications include phosphorothioate-containing oligonucleotides, methylphosphonate-containing oligonucleotides, boranophosphate-containing oligonucleotides, 2′-O-methyl-(2′-O-Me) or 2′-O-methoxyethyl oligonucleotides (2′-O)-MOE), 2′-fluoro oligonucleotides (2′-F), locked nucleic acid (LNA) oligonucleotides, peptide nucleic acids (PNAs), phosphorodiamidate morpholino oligomers (PMOs) and other chemical modifications, such as Cy3-, cholesterol-, biotin- and amino-modified oligonucleotides. Although many chemical modifications increase the stability of miRNAs, this effect may not be sufficient for in vivo applications. An efficient delivery system is generally accepted to be essential for developing miRNA-based therapeutics. Suitable carriers are divided into two types: viral vectors (1) and nonviral carriers. Nonviral carriers are divided into six categories: (2) inorganic material-based delivery systems, (3) lipid-based nanocarriers, (4) polymeric vectors/dendrimer-based carriers, (5) cell-derived membrane vesicles and (6) 3D scaffold-based delivery systems (Fu, et al., ExRNA 1, 24 (2019). https://doi.org/10.1186/s41544-019-0024-y).

ii. Vectors for Nucleic Acid Delivery Such as miRNA Delivery

As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Hamann, et al., J. Biol. Eng., 13:7 (2019) demonstrated that gene expression in hBMSCs driven by cytomegalovirus (CMV) promoter, resulted in 10-fold higher transgene expression than transfection with plasmids containing elongation factor 1 α (EF1α) or rous sarcoma virus (RSV) promoters.

Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Viral vectors can efficiently transfer genes into target cells. Various viral vectors have been constructed to mediate RNA interference (RNAi) because they can transfer genes into different tissues/organs and cause long-term gene expression. Four widely used viral vectors for miRNA delivery including adenovirus vectors, adeno-associated virus vectors, retroviral vectors, and lentivirus vectors.

Nonviral vectors for miRNA oligonucleotide delivery include inorganic material-based delivery systems, lipid based nanocarriers, polymeric vectors, for example, poly(lactide-co-glycolide) (PLGA) is a copolymer of poly lactic acid (PLA) and poly glycolic ac 3D scaffold-based delivery systemsid, chitosan, dendrimer based vectors, cell derived membrane vesicles and 3D scaffold-based delivery systems (reviewed in Fu, et al., ExRNA 1, 24 (2019). https://doi.org/10.1186/s41544-019-0024-y).

Inorganic materials, including gold nanoparticles (AuNPs), mesoporous silicon, graphene oxide and Fe3O4-mediated NPs, are widely used in nanotechnologies and have been developed as vectors to deliver miRNA. Functional groups such as thiol and amino groups can be easily attached to the surface of AuNPs, and these chemically modified AuNPs have been employed as miRNA vehicles.

Dendrimers are three-dimensional, hyperbranched globular nanopolymeric materials. A carrier denoted as NGO-PEG-dendrimers for miRNA delivery. NGO-PEG-dendrimers/anti-miRNA-21 were fabricated by conjugating PAMAM dendrimers and PEG-functionalized nanographene oxide (NGO) to 2′-O-methyl-modified mi-RNA. 3D biomaterial scaffolds can efficiently maintain the therapeutic effects of miRNA. At present, various 3D-scaffold types have been developed for miRNA delivery, including hydrogels, electrospun fibers, and other more abundantly porous or spongy 3D-scaffolds.

### 2. Pharmaceutical Compositions

Pharmaceutical compositions including EVs and/or miR296-5p and/or miR541-5p or variants thereof are also provided. Pharmaceutical compositions can be administered parenterally (intramuscular (IM), intraperitoneal (IP), intravenous (IV), subcutaneous injection (SubQ), subdermal), transdermally (either passively or using iontophoresis or electroporation), or by any other suitable means, and can be formulated in dosage forms appropriate for each route of administration.

In some embodiments, the compositions are administered systemically, for example, by intravenous or intraperitoneal administration, in an amount effective for delivery of the compositions to targeted cells.

In preferred embodiments, the compositions are administered locally, for example, by injection directly into, or adjacent to, a site to be treated or by topical administration. Typically, local injection causes an increased localized concentration of the compositions which is greater than that which can be achieved by systemic administration.

In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems: Fundamentals and Techniques (Chichester, England: Ellis Horwood Ltd., 1988 ISBN-10:0895735806), which can affect a sustained release of the material to the immediate area of the implant.

The EV compositions can be provided to the cells either directly, such as by contacting it, to or with, the cells, or indirectly, such as through the action of any biological process. For example, the vesicles can be formulated in a physiologically acceptable carrier and injected into a tissue or fluid surrounding the cells.

Exemplary dosage for in vivo methods are discussed in the experiments below. As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally, for local injection or infusion, dosage may be lower. Generally, the total amount of the active agent administered to an individual using the disclosed vesicles can be less than the amount of unassociated active agent that must be administered for the same desired or intended effect and/or may exhibit reduced toxicity.

In a preferred embodiment the compositions are administered in an aqueous solution, by parenteral injection such as intramuscular, intraperitoneal, intravenous, subcutaneous, subdermal, etc.

The formulation can be in the form of a suspension or emulsion. In general, pharmaceutical compositions are provided including effective amounts of one or more active agents optionally include pharmaceutically acceptable diluents, preservatives, solubilizers, emulsifiers, adjuvants and/or carriers. Such compositions can include diluents, sterile water, buffered saline of various buffer content (e.g., Tris-HCl, acetate, phosphate) at various pHs and ionic strengths; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

Transdermal formulations may also be prepared. These will typically be ointments, lotions, sprays, or patches, all of which can be prepared using standard technology. Transdermal formulations can include penetration enhancers. Chemical enhancers and physical methods including electroporation and microneedles can work in conjunction with this method. Typically the penetration enhancer(s) are selected such that it/they do not disrupt and/or eliminate the biological activity of the EVs.

i. Parenteral Formulations

The compounds described herein can be formulated for parenteral administration.

For example, parenteral administration may include administration to a patient intravenously, intradermally, intraperitoneally, intralesionally, intramuscularly, subcutaneously, by injection, by infusion, etc.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

### 1. Controlled Release Formulations

The parenteral formulations described herein can be formulated for controlled release including immediate release, delayed release, extended release, pulsatile release, and combinations thereof.

a. Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In embodiments wherein the formulations contains two or more agents, the agents can be formulated for the same type of controlled release (e.g., delayed, extended, immediate, or pulsatile) or the agents can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the agent(s) is controlled by diffusion of the agent(s) out of the microparticles and/or degradation of the polymeric particles by hydrolysis and/or enzymatic degradation. Suitable polymers include ethylcellulose and other natural or synthetic cellulose derivatives. Like DNA and mRNA, siRNA and miRNA can be delivered via nanocarriers. For example, Benoit et al. Biomacromolecules. 2012; 1311:3841-3849 developed a di-block co-polymer (pDMAEMA-b-p(DMAEMA-co-PAA-co-BMA)) consisting of an siRNA complexation block (pDMAEMA) and an endosomal escape block (tercopolymer of PAA, BMA, and DMAEMA) for efficient siRNA delivery.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the agent(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of agent containing microparticles. Additionally, proteins, polysaccharides and combinations thereof, which are water-soluble, can be formulated with agent into microparticles and subsequently cross-linked to form an insoluble network. For example, cyclodextrins can be complexed with individual agent molecules and subsequently cross-linked.

### 2. Method of Making Nano- and Microparticles

Encapsulation or incorporation of agent into carrier materials to produce agent-containing microparticles can be achieved through known pharmaceutical formulation techniques. In the case of formulation in fats, waxes or wax-like materials, the carrier material is typically heated above its melting temperature and the agent is added to form a mixture comprising agent particles suspended in the carrier material, agent dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, agent is added, and the molten wax-agent mixture is congealed under constant stirring as the mixture cools. Alternatively, the molten wax-agent mixture can be extruded and spheronized to form pellets or beads. These processes are known in the art. For some carrier materials it may be desirable to use a solvent evaporation technique to produce agent-containing microparticles. In this case agent and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, agent in a particulate form is homogeneously dispersed in a water-insoluble or slowly water soluble material. To minimize the size of the agent particles within the composition, the agent powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### 3. Injectable/Implantable Formulations

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods. Polyamides for nucleic acid delivery are described in U.S. Pat. No. 8,236,280

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

ii. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### 1. Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the agent and a controlled release polymer or matrix. Alternatively, the agent particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

a. Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the agent with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename EUDRAGIT t®. In further preferred embodiments, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired agent release profile can be achieved by combining low permeable and high permeable coating materials in suitable proportion.

The devices with different agent release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the agent is mixed with a wax material and either spray-congealed or congealed and screened and processed.

b. Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating an agent or an agent-containing composition with a selected coating material. The agent-containing composition may be, e.g., a tablet for incorporation into a capsule, a tablet for use as an inner core in a “coated core” dosage form, or a plurality of agent-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0) and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

iii. Topical Formulations

Suitable dosage forms for topical administration include creams, ointments, salves, sprays, gels, lotions, emulsions, and transdermal patches. The formulation may be formulated for transmucosal, transepithelial, transendothelial, or transdermal administration. The formulations can include known excipients used in topical formulations, included but not limited to sunscreens, surfactants, preservatives, desquamation agents, antiperspirants, colorants, thickeners, skin lighteners, vitamins and other therapeutically active agents in a cosmetically acceptable carrier. The compositions may further contain one or more chemical penetration enhancers, membrane permeability agents, membrane transport agents, emollients, surfactants, stabilizers, buffers, and combination thereof.

“Penetration enhancers” are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

“Preservatives” can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

Topical nucleic acid delivery using topical application, for example, topical application of naked DNA, DNA/liposomes or emulsion complex, liposomal cream, as well as physical methods such as stripping, electroporation, and micromechanical disruption methods

Methods of delivering nucleic acids (NAs) to the skin are known in the art. Physical methods include microneedle injection, microporation, electroporation, iontophoresis, sonophoresis, or passive delivery using polymeric nanoparticles; liposomes; peptides; or dendrimers. (Reviewed in Zakrewsky, et al., J. Control Release, 219:445-456 (2015)).

Intradermal injections are the simplest and most direct method for delivering NAs into the skin. Here, the barrier properties of the SC are overcome completely by injecting NAs directly into the viable tissue layers of the skin. Useful intradermal needles include microneedle arrays. Microneedle arrays comprise needles that are only 100-700 μm in length. When placed on the skin, their sharp tips allow easy insertion into the stratum corneum, while the short length ensures adequate penetration into the skin without disrupting nerves in deeper skin tissue. Microneedles can be used for delivery of nucleic acids disclosed herein.

Microporation is another technique that employs physical disruption of the SC (statun corneum) for delivery of large therapeutics or therapeutic carriers. An array of resistive elements can be placed on the skin. An electric current pulsed through the array results in localized ablation of corneocytes in contact with the array. Alternatively, erbium:yttrium-aluminum-garnet (Er:YAG) laser arrays can be used for localized ablation of the SC and epidermis. This techniques has been used to successfully deliver plasmid DNA, CpG oligonucleotides, siRNA, etc., to the skin.

Electroporation can be used to permeabilize the skin and enhance passive diffusion of agent. The mechanism of electroporation is quite different from that of electrically-induced microporation. Electrically-induced microporation utilizes electric fields to induce thermal ablation of SC microstructure creating pores in the skin. On the other hand, electroporation is the application of short duration (<0.5 s) and high intensity (<100 V) electric pulses to the skin which result in transient permeabilization of the lipid bilayers in the skin and concurrently permeabilize cell membranes of epidermal keratinocytes. Electroporation is also expected to create aqueous pores through the skin. Efficient delivery of nucleic acid molecules into skin by combined use of microneedle roller and flexible interdigitated electroporation array is disclosed in Huang, et al., Theranosties 2018: 8 (9):2361-2376.

Iontophoresis can be used to drive transport of charged drugs like NAs. Applying a continuous low intensity (<10 V) electric field at a constant current.

Liposomes have also been studied extensively for nucleic acid delivery for the treatment of skin disease.

Highly ordered spherical complexes of nucleic acids (spherical nucleic acids) have shown potential for treating skin disease due to their enhanced delivery into skin, internalization into skin cells, and protection of NAs from degradation. Specifically, gold nanoparticles coated with a dense layer of highly-ordered and covalently bound siRNA resulted in passive transport through intact mouse SC and localized exclusively in the dermis and epidermis.

The formulations can include known skin penetration enhancers. Several peptides have been identified which possess the ability to enhance transport of NAs into the skin and elicit a therapeutic response. The first of these peptides discovered using phage-display screening was TD-1 (ACSSSPSKHCG) (SEQ ID NO:55). Hsu and Mitragotri identified another peptide using phage-display screening, SPACE peptide (ACTGSTQHQCG) (SEQ ID NO:56), with the ability to not only enhance delivery of siRNA across the skin but also enhance intracellular uptake (Hsu T, Mitragotri S Proc Natl Acad Sci USA. 2011 108 (38):15816-21).

### III. Methods

1. Methods of Making Extracellular Vesicles and Compositions of EV miRNA

The disclosed compositions typically are or include extracellular vesicles derived from a sample such as plasma, obtained from a young donor subject. A “young subject” is used herein to refer to a subject having an age equivalent to 5-6 week old C57BL/6 mice. (Wang, et al., Life Sciences, 242:2020, 117242 (https://doi.org/10.1016/j.lfs.2019.117242). For example, for humans, the young donor is preferably less than about 20 years of age, for example, less than about 19, 18, 17, 16, 15, 14, 13, 12, 11, 10, 9, 8, 7, 6, 5, 4, 3, 2 or <1 yr old.

Generally, EVs can be isolated from tissue, cells, and fluid directly from a subject, including cultured and uncultured tissue, cells, or fluids, and fluid derived or conditioned by cultured cells (e.g., conditioned media). For example, exosomes are present in physiological fluids such as plasma, lymph liquid, malignant pleural effusion, amniotic liquid, breast milk, semen, saliva and urine, and are secreted into the media of cultured cells.

Methods of isolating extracellular vesicles from tissue, cells, and fluid directly from a subject, including cultured and uncultured tissue, cells, or fluids, and fluid derived or conditioned by cultured cells (e.g., conditioned media) are known in the art. See, for example, Li, Thernaostics, 7 (3): 789-804 (2017), Ha, et al., Acta Pharmaceutica Sinica B, 6 (4): 287-296 (2016), Skotland, et al., Progress in Lipid Research, 66:30-41 (2017), Phinney and Pittenger, Stem Cells, 35:851-858 (2017), each of which is specifically incorporated by reference, and describes isolating extracellular vesicles, particularly exosomes.

Extracellular vesicles, including exosomes, can be isolated using differential centrifugation, flotation density gradient centrifugation, filtration, high performance liquid chromatography, and immunoaffinity-capture.

For example, one of the most common isolation technique for isolating exosomes from cell culture is differential centrifugation, whereby large particles and cell debris in the culture medium are separated using centrifugal force between 200-100,000×g and the exosomes are separated from supernatant by the sedimenting exosomes at about 100,000×g. Purity can be improved, however, by centrifuging the samples using flotation density gradient centrifugation with sucrose or Optiprep. Tangential flow filtration combined with deuterium/sucrose-based density gradient ultracentrifugation was employed to isolate therapeutic exosomes for clinical trials.

Ultrafiltration and high performance liquid chromatography (HPLC) are additional methods of isolating EVs based on their size differences. EVs prepared by HPLC are highly purified.

Hydrostatic filtration dialysis has been used for isolating extracellular vesicles from urine.

Other common techniques for EV collection involve positive and/or negative selection using affinity-based methodology. Antibodies can be immobilized in different media conditions and combined with magnetic beads, chromatographic matrix, plates, and microfluidic devices for separation. For example, antibodies against exosome-associated antigens—such as cluster of differentiation (CD) molecules CD63, CD81, CD82, CD9, epithelial cell adhesion molecule (EpCAM), and Ras-related protein (Rab5)—can be used for affinity-based separation of exosomes. Non-exosome vesicles that carry these or different antigens can also be isolated in a similar way.

Microfluidics-based devices have also been used to rapidly and efficiently isolate EVs such as exosomes, tapping on both the physical and biochemical properties of exosomes at microscales. In addition to size, density, and immunoaffinity, sorting mechanisms such as acoustic, electrophoretic and electromagnetic manipulations can be implemented.

Methods of characterizing EVs including exosomes are also known in the art. Exosomes can be characterized based on their size, protein content, and lipid content. Exosomes are sphere-shaped structures with sizes between 40-100 nm and are much smaller compared to other systems, such as a microvesicle, which has a size range from 100-500 nm. Several methods can be used to characterize EVs, including flow cytometry, nanoparticle tracking analysis, dynamic light scattering, western blot, mass spectrometry, and microscopy techniques. EVs can also be characterized and marked based on their protein compositions. For example, integrins and tetraspanins are two of the most abundant proteins found in exosomes. Other protein markers include TSG101, ALG-2 interacting protein X (ALIX), flotillin 1, and cell adhesion molecules. Similar to proteins, lipids are major components of EVs and can be utilized to characterize them.

### 2. Methods of Treatment

Using a well-recognized mouse model of polymicrobial sepsis, developed by cecal ligation and puncture (CLP) [20], we addressed the age-associated difference in mortality following sepsis. In this report, we found that EVs from the plasma of young mice significantly reduced mortality compared to EVs isolated from older mice. The cargo of plasma EVs contains a number of molecular species and is rich in miRNAs. miRNAs are endogenous small non-coding RNAs that can bind mRNA targets and regulate gene expression by inhibiting translation or destroying the target mRNA. Several miRNAs have been found to be dysregulated in sepsis or tested as potential therapeutic targets [21-23]. We performed microRNA profiling of the plasma-derived EVs and demonstrate a sharp decline in the level of two miRNAs in the EVs with increasing age. The two miRNAs, miR296-5p and miR541-5p, suppressed LPS induced inflammation, and treatment of mice with miR246-5p reduced mortality following sepsis. We report that miR246-5p and miR541-5p are maturation-dependent factors with a potential therapeutic effect.

The disclosed compositions are administered to a subject in need thereof to treat one or more symptoms associated with inflammation.

Conditions associated with inflammation, but are not limited to, Chronic pain, traumatic brain injury, soft tissue injuries, would healing, dermatological conditions, diabetic neuropathy, Autoimmune diseases, such as rheumatoid arthritis, cardiometabolic disorders, myocarditis, stroke, Gastrointestinal disorders like inflammatory bowel disease, Crohn's disease, and ulcerative colitis, Lung diseases, such as chronic obstructive pulmonary disease (COPD) and asthma, Metabolic diseases, such as Type 2 diabetes, some cancers etc. Chronic inflammatory skin diseases are commonly encountered conditions in dermatology. They include commonly reported conditions, such as eczema, psoriasis and acne, which are associated with skin inflammation. Ohers include Seborrheic dermatitis: A common inflammatory skin condition that causes dandruff; Rosacea: A common inflammatory skin condition that causes redness across the face; Flare-ups can last for weeks or months, and small blood vessels may become visible; Rrythema nodosum: A skin condition that causes tender red nodules to form, usually on both sides of the shin. It's most common in people ages 12 to 20.; Dermatitis: A general term for conditions that cause inflammation of the skin, including contact dermatitis; poison ivy and poison oak; drug rashes, hidradenitis suppurativa, cutaneous lupus erythematosus, lichen sclerosus, alopecia areata, and vitiligo. Inflammation in the skin can range in severity from mild to severe and, in some patients, can have associated health complications.

Eczema (of which atopic dermatitis eczema is the most common type) is a common skin condition that presents as red, dry, itchy skin, often on the elbow, knee or face, but sometimes all over the body. Often associated with atopy (a predisposition to developing hypersensitivity reactions), the predominant symptom is itching.4 In some people, the skin can weep or blister and become thickened. In the chronic form of the condition there can also be altered skin pigmentation and exaggerated surface markings.2 Eczema can start at any age, but is most common in children. It is considered to be caused by a combination of genetic and environmental factors. Concurrent illness and psychological factors such as stress can also function as a trigger.

Psoriasis is a skin disease that is typically characterized by pink or red lesions which are covered with scales. These lesions are well delineated and can vary in extent and shape, and the severity of psoriasis typically follows a relapsing and remitting course. The most common form, plaque psoriasis, occurs in approximately 90% of people with the condition. Other types include guttate psoriasis and pustular forms. The cause of psoriasis is thought to be a complex interplay between genetic and environmental factors, with the immune system having an important role in the disease process.

Acne vulgaris (commonly known as acne) is a common inflammatory skin disease, which usually starts during puberty. It is characterized by a combination of comedones (blackheads and whiteheads), papules, pustules, nodules and scarring. Genetic and hormonal causes are some of the key factors that trigger the condition.

In some forms, the composition is administered to a wound site to aid in wound healing.

In some forms, the composition is administer topically to skin in a cosmetic composition, to aid in slowing skin aging.

Administration of the disclosed compositions to a subject in need thereof, is in some forms, effective to reduce intracellular pro-inflammatory cytokines such as IL-1β, TNF-α and IL-6 and inos.

In some forms the subject is septic. Sepsis is a potentially life-threatening condition that arises when the body's response to infection causes injury to its own tissues and organs. This initial stage of sepsis is followed by suppression of the immune system. Common signs and symptoms include fever, increased heart rate, increased breathing rate, and confusion. Sepsis is caused by many organisms including bacteria, viruses and fungi. Common locations for the primary infection include the lungs, brain, urinary tract, skin, and abdominal organs. Risk factors include being very young or old, a weakened immune system from conditions such as cancer or diabetes, major trauma, and burns. Sepsis requires immediate treatment. In some forms the disclosed compositions are administered to a subject diagnosed with sepsis, in combination with traditional treatments for sepsis which include intravenous fluids and antimicrobials.

Other agents can be included in the disclosed compositions, or administered in combination therewith, such as one or one or more anti-inflammatory agents such as steroid and non-steroid drugs. Suitable steroids agents include glucocorticoids, progestins, mineralocorticoids, and corticosteroids. Small molecule steroidal anti-inflammatories include prednisone, dexamethasone, cortisone, loteprednol, triamcinolone acetonide, fluocinolone acetonide, fluorometholone, and fluticasone. Exemplary immune-modulating drugs include cyclosporine, tacrolimus and rapamycin. In some embodiments, anti-inflammatory agents are biologic drugs that block the action of one or more immune cell types such as T cells, or block proteins in the immune system, such as tumor necrosis factor-alpha (TNF-alpha), interleukin 17-A, interleukins 12 and 23.

The frequency of administration of a method of treatment can be, for example, one, two, three, four or more times daily, weekly, every two weeks, or monthly. In some embodiments, the composition is administered to a subject once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the frequency of administration is once, twice or three times weekly, or is once, twice or three times every two weeks, or is once, twice or three times every four weeks. In some embodiments, the composition is administered to a subject 1-3 times, preferably 2 times, a week.

In some embodiments, the effect of the disclosed compositions and methods on a subject is compared to a control. For example, the effect of the composition on a particular symptom, pharmacologic, or physiologic indicator (including those mentioned above and elsewhere herein) can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known in the art, such as one of those

The present invention will be further understood by means of the following non-limiting examples.

### EXAMPLES

### Materials and Methods

### Mouse Strain and Animal Housing

All animal care and use procedures were performed according to the protocol approved by the Institutional Animal Care and Use Committee of Augusta University and were in compliance with the “Guide for the Care and Use of Laboratory Animals” published by the National Institutes of Health. Male C57BL/6 mice of ages 5-6 weeks, 14-16 weeks, 12 months, and 23-26 months were used in this study. The mice were purchased from Charles River Laboratory or through the National Institute on Aging and housed in the Augusta University vivarium.

### Mouse Model of Polymicrobial Sepsis

As described previously, polymicrobial sepsis was induced by cecal ligation and puncture (CLP) [20]. In brief, mice were anesthetized with isoflurane (2.5%), the cecum was ligated at ˜40% length from the distal pole, punctured through with a 22-gauge needle, and gently squeezed to take out a small amount of fecal material. The abdominal incision was closed in two layers. The sham-operated mice underwent general anesthesia and laparotomy but not the CLP. Carprofen was administrated subcutaneously for pain control immediately before and every 12 hours for 24 hours after surgery. After the surgery, prewarmed saline (4 ml/kg body weight) was injected subcutaneously. Sepsis score was monitored as described in [24].

### EV Isolation and Characterization

Plasma EVs were isolated by column chromatography using Extracellular Vesicle Size Exclusion Chromatography Columns (STEMCELL technologies, Cambridge, MA). In brief, plasma was collected and diluted with an equal volume of Ca2+/Mg2+-free Dulbecco's PBS (D-PBS) and then centrifuged at 300 g for 10 minutes to remove cell debris and protein aggregates. The supernatant was centrifuged at 1200 g for 20 minutes, followed by 10,000 g for 30 minutes. The supernatant was loaded to the top of the column and 100 μL fractions were collected. EVs are typically eluted from fractions 9-14. EVs were quantified using a nano tracking system, Particle Metrix (Zeta View, Ammersee Germany), and 9-14 fractions were found to be consistently enriched for EVs. EV proteins were measured with the Bradford method using Micro BCA Protein Assay Kit (Thermo scientific). Immuno-electron microscopy was done at the histology core to detect exosomal surface markers, CD9 and CD63.

### In Vivo Treatments

To determine the effect of plasma factors from mice of varying ages on sepsis outcome, plasma EVs (10⁸ particles/dose) were injected by tail vein after at 2 h, 24 h and 48 h post-CLP surgery. The mice were scored for sepsis daily [24]. To determine the effect of miR296-5p, the miRNA mimic or negative control miRNA was administered 24 hours before and at 2 hours after the CLP surgery intraperitoneally (5 ug/Kg) as described previously [21]. The recipient mice were monitored for sepsis severity and survival.

### SPECT/CT Imaging

Single-photon emission computed tomography (SPECT) images were acquired on a NanoScan™ (Mediso, Budapest, Hungary) using Indium-111 trapped EVs. EVs were injected through tail vein and the images were acquired after two hours to assess tissue-distribution of the EVs. Multiplanar SPECT-CT images were reconstructed for display using manufacturer-supplied software (Nucline, Mediso). All SPECT images were formatted in Analyze format and multiplanar reconstruction.

RNA and DNA Isolation and miRNA Sequencing.

Total RNA in tissues was isolated using TRIzol reagent according to the manufacturer's protocols (Thermal Fisher, Carlsbad, CA; 4478545). cfDNA was isolated using Plasma/Serum Cell-Free Circulating DNA Purification Micro Kit (NORGEN, Cat.55500). Total miRNA was extracted using the PureLink total miRNA Isolation Kit (Invitrogen, Carlsbad, CA; K157001). cDNA from total RNA was synthesized using ImProm-II™ Reverse Transcription System (Promega, Madison, WI). cDNA templates were prepared from EV miRNA using Advanced miRNA cDNA Synthesis Kit (TaqMan®, A28007). Total RNA in EVs was extracted using the Total Exosome RNA Isolation Kit (Invitrogen, 4478545). RNA quality was assessed using the Agilent 2100 Bioanalyzer. miRNA Library was prepared by using Illumina TruSeq stranded mRNA kit and sequencing was carried out at Novogene (Durham, NC).

### Bioinformatics and Statistical Analysis

The raw sequencing reads were analyzed with bioinformatics analysis pipeline using FastQC for quality control, Cutadapt for adapter trimming, and STAR for alignment, and mm10 mouse genome for references. Differential expression analyses were performed on the miRNA count using DESeq2 package in R. The p-values were adjusted with false discovery rate, and significance threshold of 0.05 was used.

Cell Culture and Transient Transfection of miRNA Mimics

Primary MEF cells were generated from mouse embryos at 12-13 days [25]. MEF cells were cultured in DMEM (Dulbecco's Modified Eagle's Medium, Gibco 11965-092) supplemented with 10% fetal bovine serum and penicillin/streptomycin. 0.5×10⁵ MEF cells were seeded in 12-well plates 18-24 h prior to transfection. Synthetic miRNA 296-5p and miRNA541-5p mimics (from Qiagen) were transfected into cells following the Lipofectamine™ RNAiMAX Transfection protocol (Invitrogen 13778100). Twenty-four hours later, cells were treated with LPS (10 ng/ml) and cultured for an additional 24 h. MEF cells were co-treated with EV from young, mature and aged mice and either LPS or doxorubicin. LPS-treated cells were washed twice with PBS and lysed in TRIzol reagent (Invitrogen, #1196018). Doxorubicin (250 nm)-treated cells were collected and tested for senescence markers p16 and p21 by flow cytometry.

### Reverse Transcription-Real Time Polymerase Chain Reaction (RT-PCR)

RT-PCR was performed using standard procedures using real time PCR. The expression levels were normalized to β actin. qPCR was performed using a Real-Time PCR instrument (Stratagene Mx3000p: Agilent Technologies) with SYBR Advantage qPCR mix. The thermocycling conditions were as follows: initial denaturation for 30 sec at 95° C., followed by 40 cycles of 10 sec at 95° C., and 30 sec at 60° C. Relative mRNA expression was calculated using the 2-ΔΔCq method.

TABLE 1
Primer sequences for real time RT-PCR.

| Genes | Forward Primer | Reverse Primer |
|---|---|---|
| IL-6 | CCGGAGAGGTGACT | TTCTGCAAGTGCAT |
| | TCACAG | CATCGT |
| | (SEQ ID No: 1) | (SEQ ID No: 2) |
| MCP-1 | CCC ACT CAC CTG | TCT GGA CCC ATT |
| | CTG CTA CT | CCT TCT TG |
| | (SEQ ID No: 3) | (SEQ ID No: 4) |
| IL-1α | TCTCAGATTCACA | AGAAAATGAGGTC |
| | ACTGTTCGTG | GGTCTCACTA |
| | (SEQ ID No: 5) | (SEQ ID No: 6) |
| P53 | TGGCCATCTACAAGA | ATCGGAGCAGCGC |
| | AGTCACAG | TCATG |
| | (SEQ ID No: 7) | (SEQ ID No: 8) |
| P21 | GTTCCGCACAGGAGC | ACGGCGCAACTGC |
| | AAAGT | TCAC |
| | (SEQ ID No: 9) | (SEQ ID No: 10) |
| β-actin | AGTACCCCATTGAAC | AATGCCAGTGGTAC |
| | ACG | GACC |
| | (SEQ ID No: 11) | (SEQ ID No: 12) |

### LDH Cytotoxicity Assay

MEF cells were plated onto 96-well plates at a density of ˜9,000 cells/well. The next day, the cells were treated with 10⁸ EV particles. The cells were incubated with EVs for 24 h. After incubation, the cells were washed with PBS and treated with LPS (100 ng/ml) for 24 h. After treatment, cell death was evaluated using a CyQUANT™ LDH Cytotoxicity Assay Kit (Invitrogen, Waltham, MA, USA). Cytotoxicity was calculated using the formula:

$$[ ( EV - treated LDH activity - spontaneous LDH activity ) \text{} / ( maximum LDH activity - spontaneous LDH activity ) ] \times 100 .$$

### Flow Cytometry

MEF cells were treated with doxorubicin (250 nM) or vehicle, incubated with EVs, and collected for flow cytometry. To evaluate senescence in MEF cells by flow cytometry, cells were incubated with the following antibodies: p16-AF647 and p21-AF488 (Abcam) after treating with fixation/permeabilization solution (BioLegend, San Diego, CA). The percentage of p21 and p16 positive cells and the MFI of p21 and p16 expression were analyzed using FlowJo (version 10.4.2).

### In Vitro Scratch Assay

Rat-Pulmonary microvascular vascular endothelial cells (PMVEC) were seeded in a 12-well plate at a density of 1×10⁵ cells/well and cultured until they reached 80% of confluence. Before scratching the cell monolayer, the base of the wells was marked with horizontal reference lines (two parallel lines approx. 0.3 cm apart with a fine tip marker) to ensure uniform scratching and to obtain the same field for each image acquisition time point. The cell monolayer was then scratched with a 1000 μL sterile pipette tip using the pointed end. The detached cells were then washed away with PBS (1×). 1 mL of complete medium (DMEM+10% FBS+1×Pen-Strep, Gibco) with EVs were then added to the cells. To test the effect of miRNA mimics (miR296-5p and miR 541-5p) the PMVECs were transfected with respective miRNAs 24 hr prior to scratching. The effect of antagomirs was assessed by first incubating the EVs from young animals with respective antagomirs and Lipofectamine for 90 minutes at room temperature and then added to the cells. micrographs of scratched regions of interest were captured using an Echo-revolve inverted microscope on a 4× objective at 0 hr, 24 hr and 48 hr post-scratch. From each well at least 2-3 pictures were captured for the complete scratch area over the horizontal diameter of the well. In the case of a non-uniform pattern of cell growth in the scratch, multiple images were taken for evaluation. The images from the corners of the well were omitted from the analysis since these images contained the blunt ends of the scratches and were highly variable and inconsistent due to the increased cell density. The total scratch area for each of the four technical replicates was obtained by combining the scratch areas of 2-3 fields of view taken per replicate. The MRI Wound Healing Tool was used to analyze scratch assays as a plugin to ImageJ software (Github).

### Statistical Analysis

Statistical analyses were performed using GraphPad Prism. The differences between two groups were assessed by two tailed t-test. Multiple group comparisons were made by one-way ANOVA. A 2-way ANOVA was used for analysis of results of scratch assay. p<0.05 was considered significant.

### Results

Age-Associated Survival after CLP

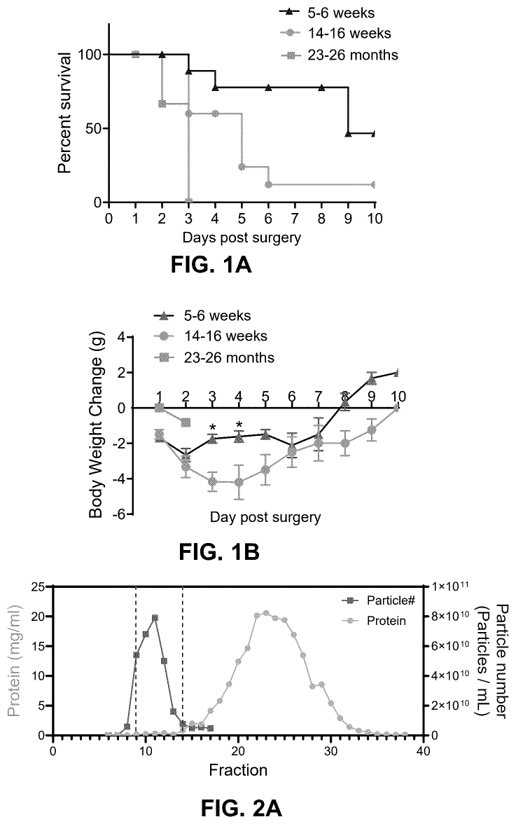

The effect of age on the survival of mice following CLP surgery was assessed using mice of three different age groups: young (5-6 weeks), mature (14-16 weeks), and aged (23-26 months) (FIGS. 1A and B). The Kaplan-Meier survival curves for each of the three age groups subjected to CLP showed progressive increase in mortality with age, demonstrating a strong influence of age on the outcome following sepsis in this experimental model. While a majority of the mice (80%) in the mature group died within six days after CLP, all mice in the aged group died within three days (FIG. 1A). The 10-day survival rate among the younger mice was significantly higher than in the mature (p=0.0375) or the aged mice (p=0.0003). The change in body weight of the surviving mice in the younger group also remained less than that of mature mice, further demonstrating that young mice were more tolerant to CLP-induced sepsis.

Plasma EV Size and Number, and Mt-DNA Levels Vary with Age

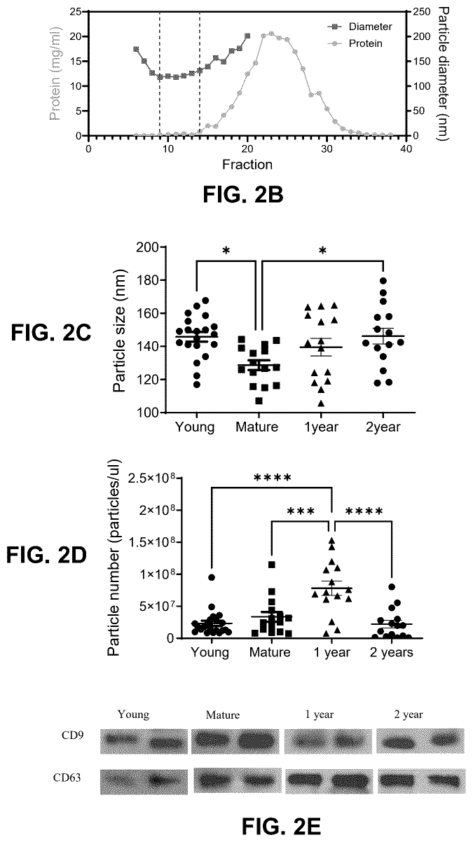

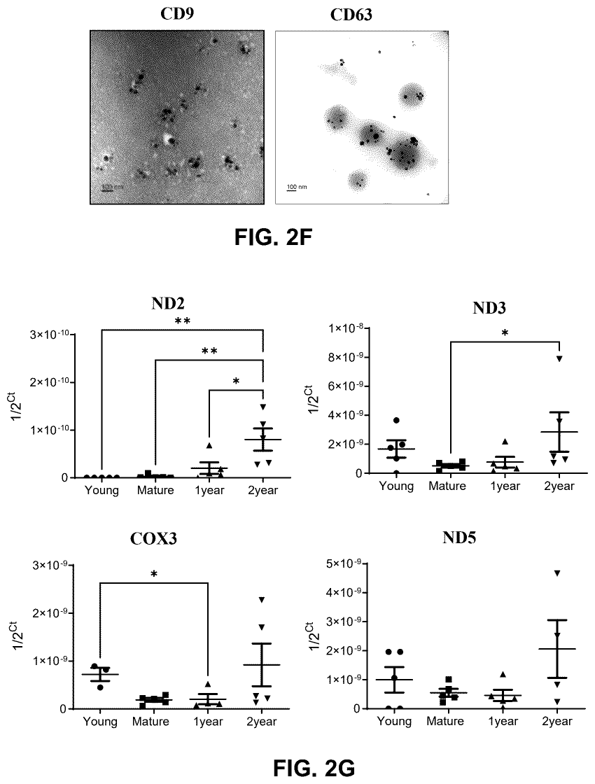

EVs were isolated from the blood plasma using size exclusion chromatography and were characterized by a nanoparticle tracking analyzer (NTA) for count and size, western blot for cell surface markers, and transmission electron microscopy for size and specificity. As shown in FIGS. 2A and 2B, fractions 9-14 had the most EVs with least plasma protein content. These fractions were pooled together prior to use. The diameter of the isolated EVs was in the range of 100-200 nm, and the concentration was ˜2-8×10¹⁰ particles/ml/fraction (FIG. 2A, B). The average size of EVs from the young and aged mice was significantly higher than those isolated from the mature mice (FIG. 2C). However, the number of EVs per microliter from one-year-old mice was higher than that in other groups (FIG. 2D). In addition, CD9 and CD63, the exosome protein markers, were observed on EVs isolated from all four age groups (FIG. 2E). Transmission electron microscopy was used to further confirm the size, morphology, and markers of the EVs (FIG. 2F).

Based on the prevailing hypothesis that there is an age-associated increase in circulating cell-free mitochondrial DNA (ccf-mtDNA), studies tested whether there is a change in the level of mtDNA in the EVs with aging. cef-mtDNA fragments are DAMPS and were demonstrated to cause inflammation and tissue damage [26]. Four different gene segments on mitochondria, ND2, ND3, ND5, and COX3, from 10⁷ EV particles were amplified and found a significantly progressive increase in ND2 with age (FIG. 2G). mtDNA levels were highest in the EVs isolated from the 2-year-old mice, among all the age groups tested.

Influence of Donor Age on EV-Mediated Effect on Survival in Mice with Sepsis.

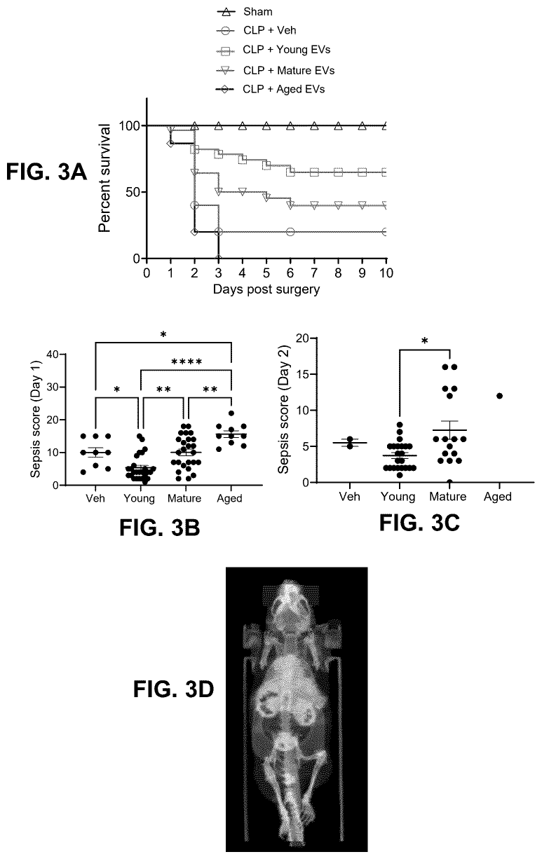

The EV cargo contains diverse biomolecules derived from various cell types in the organism and have been observed to have a cytoprotective effect for plasma and MSC-derived EVs in experimental animals [27]. Therefore, studies tested whether EV donor age influences the mice's survival outcomes after CLP surgery. The recipient mice received 10⁸ particles/dose of EVs through the tail vein at 2, 24, and 48 hours after CLP. In the group of mice that received young EVs, 68% survived, and their 10-day survival rate was significantly higher in comparison with the vehicle (20%; p=0.0009), mature EV (43%; p=0.045), or aged EV group (0) %; <0.0001) (FIG. 3A). For a quantitative assessment of sepsis severity, a previously established 24-point sepsis scoring method was used, as detailed in the methods section. This allowed evaluation of the sepsis severity at different time points by monitoring spontaneous activity, response to touch and auditory stimuli, posture, respiration rate, quality, and appearance (FIG. 3B-C). At 24 hours after the surgery, the CLP mice receiving young EVs had lower sepsis scores than those receiving mature EVs, aged EVs, or the vehicle. On day 2, the composite sepsis severity score in the group that received young EVs was continued to be lower than the recipients of mature EVs, while most of the mice that received aged EVs or vehicle did not survive beyond 2 days (FIG. 3C). FIG. 3D illustrates the propensity of IV-injected EVs to home to most of the internal organs.

EVs from Young Mice Protect Cells from Inflammation, Senescence, and Death.

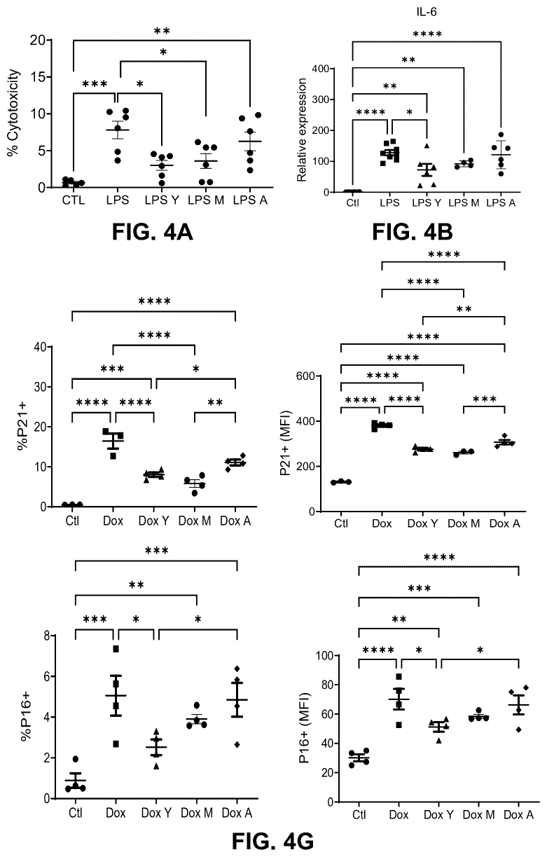

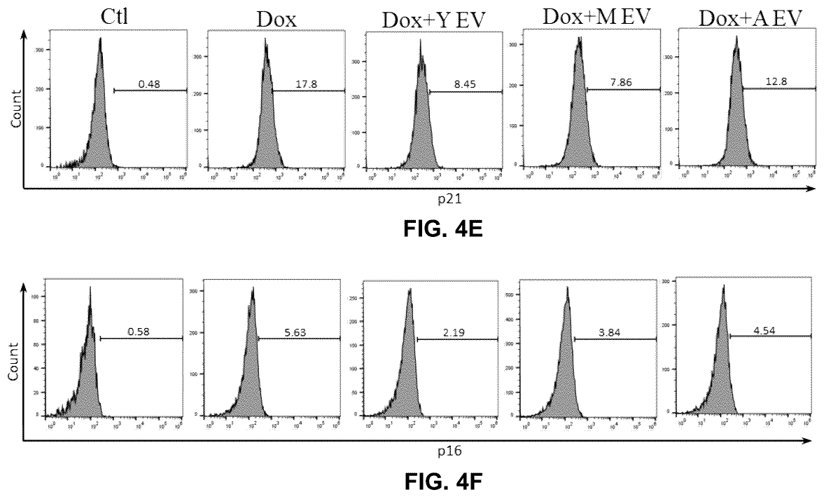

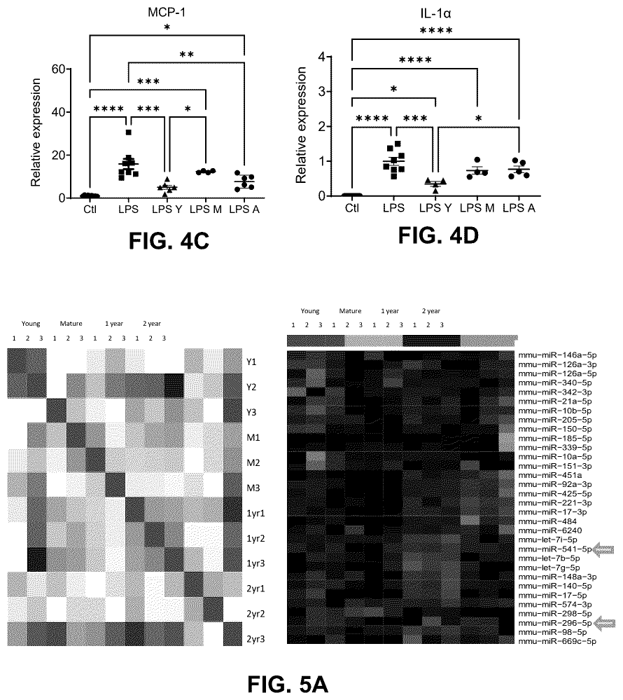

The age-associated propensity to inflammation, cellular senescence, and cell death is well described. Sepsis also alters the systemic and tissue microenvironment, triggering immune dysregulation and cell death. To determine whether young EVs confer protection from cytotoxicity and inflammation, MEF cells were treated with LPS and co-cultured them in the presence or absence of EVs isolated from the young, mature, or aged mice. The cytotoxicity, as measured by LDH release, was significantly reduced when the cells were treated with young EVs (FIG. 4A). Inflammatory markers IL-6, IL-1 . . . , and MCP-1 gene expression were also reduced in the MEF treated with young EVs (FIG. 4B-D). Senescence was induced in MEF cells by treating them with doxorubicin and treated with the EVs. While doxorubicin treatment increased p21 expression in the MEF cells, the cells co-treated with young EVs demonstrated downregulation of both p21 and p16, as evidenced by the reduced number and mean fluorescence intensity of p21 and p16 positive cells by flow cytometry (FIG. 4E-G).

Age-Associated Changes in miRNA Profile in the EVs.

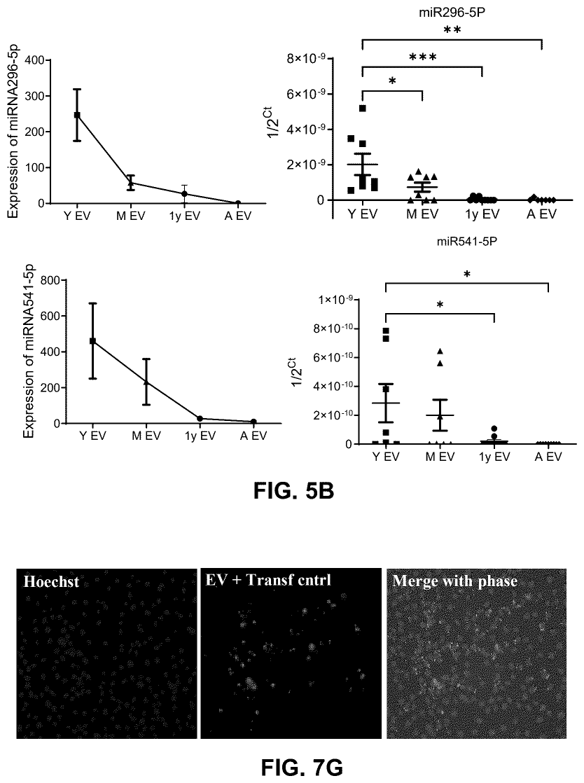

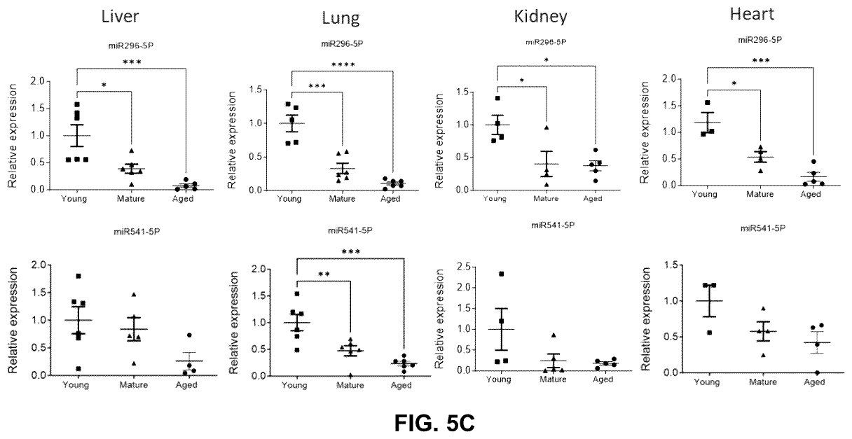

A major constituent of EVs is miRNA, and by sequencing, the cargo in the plasma EVs derived from mice of four different age groups, ranging from 5 weeks to 2 years, was profiled. The donor age of the EVs correlated well with the miRNA expression pattern, suggesting an age-associated miRNA signature in the plasma-derived EVs (FIG. 5A). 499 miRNAs were identified in the cargo of plasma EVs derived from mice of at least one age group. The expression level of 32 miRNAs showed a significant difference in one or more age groups when compared to the levels of respective miRNAs from the young EV group. These miRNAs are shown in a heatmap in FIG. 5A (right panel). When miRNAs that showed a progressive decline or increase with age were investigated, the studies showed that levels of two miRNAs, miR296-5p and miR 541-5p, to be sharply declining with age (FIG. 5A right panel). This result was further confirmed by PCR amplification of both miRNAs from the respective EVs; the results showed a consistent decrease with progressive aging (FIG. 5B).

Subsequent studies tested age and tissue-specific expression of the miRNAs, miR296-5p and miR 541-5p, in the liver, lung, kidney, and heart of young, mature, and aged mice to determine their age- and tissue-dependent changes. The expression of both miR296-5p and miR 541-5p were highest in the young mice, compared to the mature or the aged mice (FIG. 5C). The progressively reducing levels of the two miRNAs suggest that these two miRNAs are regulated by the aging process in critical tissues.

The Effect of miR296-5p and 541-5p on Inflammation

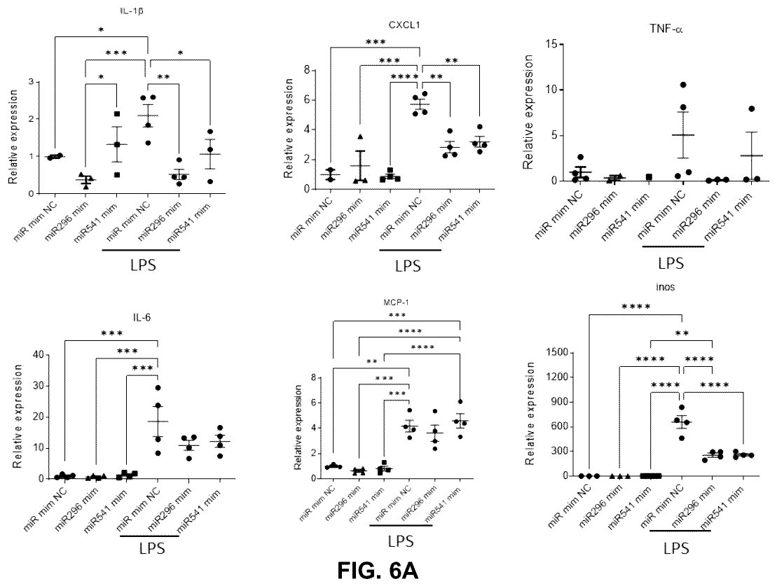

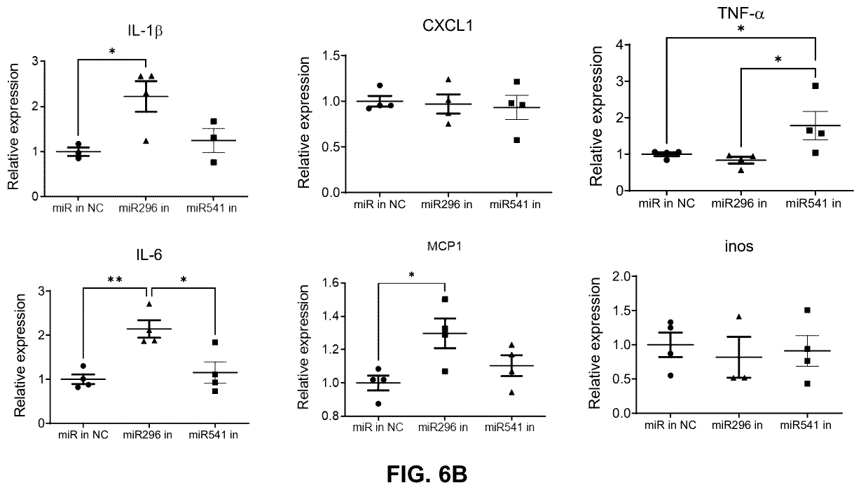

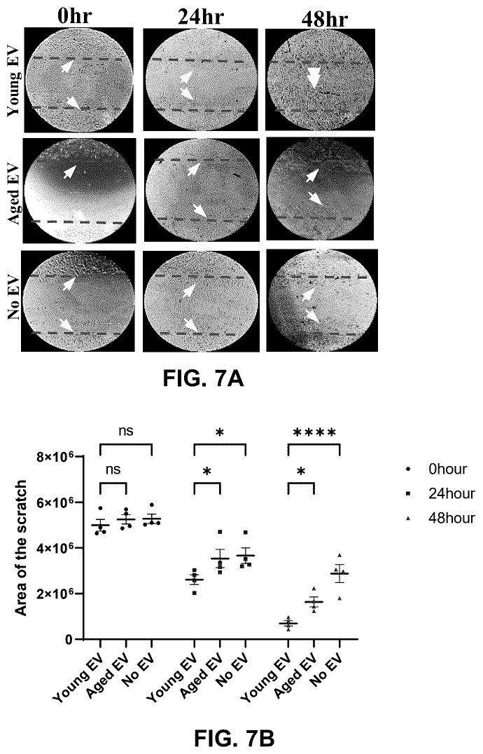

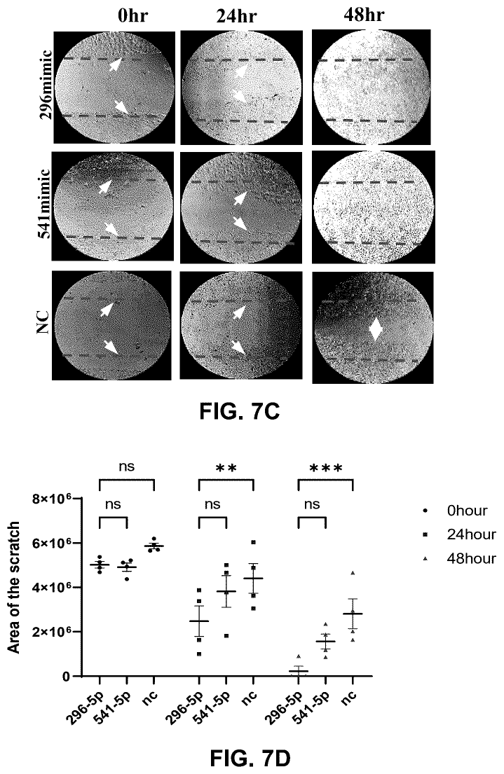

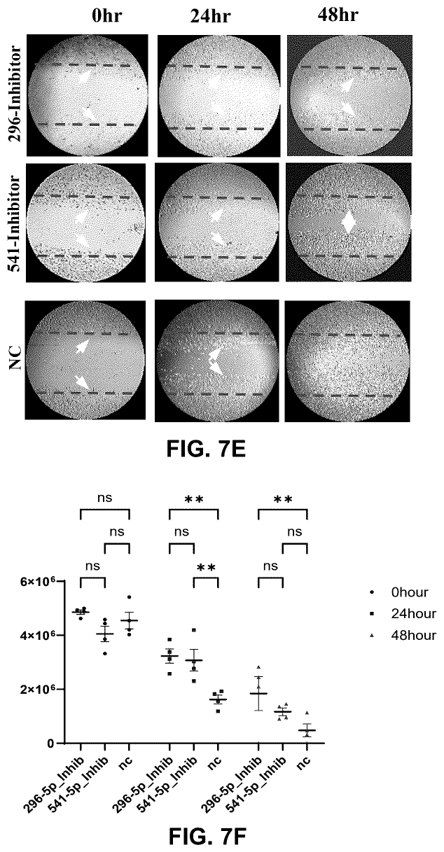

To further test whether the anti-inflammatory effect observed with the young EVs were contributed by any of the two miRNAs identified, MEF cells were transfected with the miRNA mimics and treated with LPS. While transfection of MEF with the miRNAs had an anti-inflammatory effect, LPS treatment of the mimic transfected cells demonstrated a more profound inhibition of the inflammatory phenotype. (FIG. 6A). miR296-5p or miR541-5p mimics markedly reduced the gene expression of intracellular pro-inflammatory cytokines such as IL-1β. TNF-α and IL-6 and inos in the MEF cells (FIG. 6A). The effect was reversed when MEF cells were transfected with antagomirs of miR296-5p and miR541-5p (FIG. 6B). When either of the two miRNAs were inhibited with the antagomirs, the gene expression as assessed by real-time PCR showed significant expression of several proinflammatory genes. While miR296-5p inhibition resulted in an increased expression of IL-1□, IL-6, and MCP1, inhibition of miR541-5p resulted in significant (over) expression of TNF-□ demonstrating that both miR296-5p and miR541-5p possess anti-inflammatory properties (FIG. 6B).

Young EVs Containing miR-296/541-5p May Improve Wound Healing in PMVECs

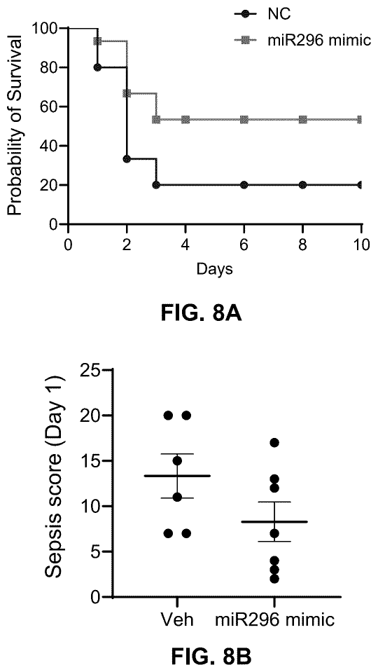

To compare and assess the regeneration capability of young and aged EVs, and the two miRNAs as their functional component aiding in regeneration, PMVECs were treated with young and aged EVs. The EVs derived from the plasma of young mice showed significant and faster healing of the scratched wound than the EVs derived from aged mice, in a span of 48 hours. Similarly, our experiments showed that when the cells were treated with 296-5p or 541-5p mimics the scratched surface showed significantly higher wound closing compared to that treated with negative control. Furthermore, the inhibition of 296-5p by the antagomir was more effective in slowing the wound healing process in comparison to 541-5p antagomir at 48 hours. (FIG. 8A-H).

miR296-5p Rescues Mice from CLP-Induced Sepsis

Our next goal was to determine whether miR296-5p and miR541-5p can rescue mice from death after CLP surgery. miRNA-296-5p miR541-5p or control miRNA was delivered intraperitoneally 24 hours before and two hours after the CLP surgery. Our preliminary experiments with 5 mice each showed similar effect for miR541-5p and the control miRNA. So further studies investigated the effect of miR296-5p on sepsis-induced mortality. Mice were monitored for 10 days and the miR296-5p recipient mice showed significantly improved sepsis score and survival (FIG. 8A-B).

### DISCUSSION

Aging systems gradually lose agility, with a decline in physiological function. This deceleration of growth and acceleration of aging begins early in life, as it has been shown that age-associated changes in gene expression originate during the juvenile period of growth deceleration. Furthermore, the susceptibility to injury-induced death increases with age [29, 30]. This report, for the first time, shows that when sepsis was induced in mice ages 5 weeks to 2 years, the mice at or before puberty were more resilient to the adverse sequel following the CLP surgery. While all the mice in the aged group died within three days, half of the mice in the 5-6-week-old group survived after CLP. Though the mean survival duration of mature (14-16-week-old) mice was longer than that of the aged mice, it was significantly shorter than that of the young mice. This experiment demonstrates that very young animals are more resilient in responding to injury, infection, and stress. However, the causative factors for the resiliency of the young mice are unknown and the factors in the young blood responsible for the reparative effect in the aged systems remain unknown.

Recently, some reports have defined proteins such as platelet factor 4 (PF4) and Klotho as rejuvenating factors. In one of the studies, when aged male mice were systemically treated with young blood plasma or platelet fraction, profound gene expression changes associated with immune regulation and nervous system development were observed in the brain; however, these changes were not observed with blood plasma or platelet fraction from the aged mice [14]. Systemic injection of PF4, which was found to be elevated in the young plasma, into aged mice reduced inflammation in the hippocampus and improved cognition [14]. Simultaneously, another group found that systemic increase in PF4 improved age-related cognitive impairments and hippocampal neurogenesis [15]. Furthermore, the same group showed that exercise activates platelets that are essential for exercise-induced neurogenic processes. In a different study, when mice were treated with PF4, synaptic plasticity and cognitive function were improved in the young mice and PF4 reduced cognitive defects in the aged brain [16]. Though they found similar improvement with Klotho suggesting PF4 mediates the effect, the same was also observed in PF4 deficiency, speculating yet other mediators in the young blood [16]. The presence of Klotho in extracellular vesicles from young serum was previously found to regulate skeletal muscle regeneration in the aged mice [12]. EVs from the young blood were a focus of similar studies which showed that the young EVs exhibited GSTM2 activity, reversed oxidative stress, and reduced senescence-associated tissue damage [7, 11].

The data herein shows that when mice were injected with EVs derived from blood plasma, the donor age of the EVs is a major determining factor in determining the outcome after sepsis. The mortality after sepsis induction was the lowest among the recipients when the EV donors were at or prior to pubertal age. The EVs from the young mice reduced cytotoxicity, suppressed markers of senescence, and downregulated inflammatory genes in MEF cells treated with LPS. The reduced level of mtDNA in the young plasma EVs also supports their reduced inflammatory effect when compared to EVs from the aged mice. Our scratch wound experiments using rat PMVECs show that young EVs also improve regeneration in comparison to EVs from aged mice.

To determine the molecular identities of young plasma factor(s) contributing to improved survival after sepsis, the miRNA content of the EV cargo was analyzed. While a number of miRNAs were identified in the EVs, only two miRNAs showed a progressive decline with age, miRNA-296-5p and miRNA-541-5p. While both of them demonstrated anti-inflammatory effect, the antagomirs of both miRNA-296-5p and miRNA541-5p were able to induce significant expression of inflammatory cytokines demonstrating that miRNA 296-5p may be a potent inhibitor of inflammation. The lack of a similar increase of inflammatory cytokines in MEF cells treated with the mimics of the two miRNAs and the ability of the mimics to suppress LPS-induced inflammation in MEF cells further support the inference that miRNA-296-5p and miRNA541-5p have anti-inflammatory-effects, miRNA-296-5p being more effective. Interestingly enough, both miRNAs are expressed in most tissues, with a sharp decline in expression with progressing age, or more specifically post maturation.

The present experiments show that the presence of miRNA-296-5p and miRNA-541-5p mimics aid the regeneration process in young EVs. Interestingly, their inhibition also decelerated the wound healing. A recent study showed that transplanting aged human skin onto 2-month-old young SCID/beige mice rejuvenated the xenotransplants. However, when the same was transplanted to 14-month-old mice, no rejuvenation effect was observed in the old human skin [31]. The rejuvenation effect was followed by a significant increase in VEGF expression in the young, while the rejuvenation effect of the recipient mouse was lost with progressing age. Furthermore, VEGF has been previously proposed to be a key driver of human organ rejuvenation and its level was found to be reduced with age [31]. On the contrary, VEGF promotes vascular permeability, an important pathophysiological mechanism of sepsis, with a positive correlation between VEGF levels and severity/mortality [32]. Importantly, the VEGF has been previously shown to increase the expression of miR-296, also termed by some as angiomiR. miR-296 was reported to be elevated during angiogenesis, reduces the substrate that degrades VEGFR2 and PDGFR-β and promote angiogenesis [33-35]. The inhibition of miR-296 abolished VEGF-induced angiogenesis suggesting a role for this miRNA in angiogenesis and rejuvenation. However the present studies show that that miR-296-5p may be the effector of the rejuvating function exhibited by VEGF or the effect is VEGF-independent, as the studies did not show an increase in VEGF when MEF cells were treated with miR296-5p (data not shown).

In summary, the present studies demonstrate that EVs from very young mice have a reparative effect on wound healing and sepsis, and the reparative factors are likely maturation-dependent. The observation that miRNA-296-5p is a plasma EV constituent that significantly reduces with age and can reduce mortality following sepsis suggests a therapeutic potential for this miRNA in sepsis and possibly aging and age associated co-morbidities.

### REFERENCES

[1] M. V. Blagosklonny, Rapamycin treatment early in life reprograms aging: hyperfunction theory and clinical practice, Aging (Albany NY) 14(20) (2022) 8140-8149.
[2] J. A. Baur, D. A. Sinclair, Therapeutic potential of resveratrol: the in vivo evidence, Nat Rev Drug Discov 5(6) (2006) 493-506.
[3] C. Shade, The Science Behind NMN-A Stable, Reliable NAD+Activator and Anti-Aging Molecule, Integr Med (Encinitas) 19(1) (2020) 12-14.
[4] L. Rochette, G. Dogon, E. Rigal, M. Zeller, Y. Cottin, C. Vergely, Growth differentiation factor 11: A proangiogenic drug as a potential antiaging regulating molecule, Arch Cardiovasc Dis 116(1) (2023) 41-46.
[5] L. K. Rogers, P. A. Lucchesi, Stress adaptation and the resilience of youth: fact or fiction?, Physiology (Bethesda) 29(3) (2014) 156.
[6] M. Khalil, G. Alawwa, F. Pinto, P. A. O'Neill, Pediatric Mortality at Pediatric versus Adult Trauma Centers, J Emerg Trauma Shock 14(3) (2021) 128-135.
[7] X. Chu, K. Subramani, B. Thomas, A. V. Terry, Jr., S. Fulzele, R. P. Raju, Juvenile Plasma Factors Improve Organ Function and Survival following Injury by Promoting Antioxidant Response, Aging Dis 13(2) (2022) 568-582.
[8] M. J. Conboy, I. M. Conboy, T. A. Rando, Heterochronic parabiosis: historical perspective and methodological considerations for studies of aging and longevity, Aging Cell 12(3) (2013) 525-30.
[9] X. Chu, R. P. Raju, The tale of young blood rejuvenating the old, Aging (Albany NY) 15(17) (2023) 8533-8534.
[10] M. Sinha, Y. C. Jang, J. Oh, D. Khong, E. Y. Wu, R. Manohar, C. Miller, S. G. Regalado, F. S. Loffredo, J. R. Pancoast, M. F. Hirshman, J. Lebowitz, J. L. Shadrach, M. Cerletti, M. J. Kim, T. Serwold, L. J. Goodyear, B. Rosner, R. T. Lee, A. J. Wagers, Restoring systemic GDF11 levels reverses age-related dysfunction in mouse skeletal muscle, Science 344(6184) (2014) 649-52.
[11] J. A. Fafian-Labora, J. A. Rodriguez-Navarro, A. O'Loghlen, Small Extracellular Vesicles Have GST Activity and Ameliorate Senescence-Related Tissue Damage, Cell Metab 32(1) (2020) 71-86 e5.
[12] A. Sahu, Z. J. Clemens, S. N. Shinde, S. Sivakumar, A. Pius, A. Bhatia, S. Picciolini, C. Carlomagno, A. Gualerzi, M. Bedoni, B. Van Houten, M. Lovalekar, N. F. Fitz, I. Lefterov, A. Barchowsky, R. Koldamova, F. Ambrosio, Regulation of aged skeletal muscle regeneration by circulating extracellular vesicles, Nat Aging 1(12) (2021) 1148-1161.
[13] S. Buchanan, E. Combet, P. Stenvinkel, P. G. Shiels, Klotho, Aging, and the Failing Kidney, Front Endocrinol (Lausanne) 11 (2020) 560.
[14] A. B. Schroer, P. B. Ventura, J. Sucharov, R. Misra, M. K. K. Chui, G. Bieri, A. M. Horowitz, L. K. Smith, K. Encabo, I. Tenggara, J. Couthouis, J. D. Gross, J. M. Chan, A. Luke, S. A. Villeda, Platelet factors attenuate inflammation and rescue cognition in ageing, Nature 620(7976) (2023) 1071-1079.
[15] O. Leiter, D. Brici, S. J. Fletcher, X. L. H. Yong, J. Widagdo, N. Matigian, A. B. Schroer, G. Bieri, D. G. Blackmore, P. F. Bartlett, V. Anggono, S. A. Villeda, T. L. Walker, Platelet-derived exerkine CXCL4/platelet factor 4 rejuvenates hippocampal neurogenesis and restores cognitive function in aged mice, Nat Commun 14(1) (2023) 4375.
[16] C. Park, O. Hahn, S. Gupta, A. J. Moreno, F. Marino, B. Kedir, D. Wang, S. A. Villeda, T. Wyss-Coray, D. B. Dubal, Platelet factors are induced by longevity factor klotho and enhance cognition in young and aging mice, Nat Aging 3(9) (2023) 1067-1078.
[17] A. Laviano, Young blood, N Engl J Med 371(6) (2014) 573-5.
[18] G. S. Martin, D. M. Mannino, M. Moss, The effect of age on the development and outcome of adult sepsis, Crit Care Med 34(1) (2006) 15-21.
[19] E. H. A. Michels, J. M. Butler, T. D. Y. Reijnders, O. L. Cremer, B. P. Scicluna, F. Uhel, H. Peters-Sengers, M. J. Schultz, J. C. Knight, L. A. van Vught, T. van der Poll, M. consortium, Association between age and the host response in critically ill patients with sepsis, Crit Care 26(1) (2022) 385.
[20] L. Cai, A. S. Arbab, T. J. Lee, A. Sharma, B. Thomas, K. Igarashi, R. P. Raju, BACH1-Hemoxygenase-1 axis regulates cellular energetics and survival following sepsis, Free Radic Biol Med 188 (2022) 134-145.
[21] M. P. Dragomir, E. Fuentes-Mattei, M. Winkle, K. Okubo, R. Bayraktar, E. Knutsen, A. Qdaisat, M. Chen, Y. Li, M. Shimizu, L. Pang, K. Liu, X. Liu, S. Anfossi, H. Zhang, I. Koch, A. M. Tran, S. Mohapatra, A. Ton, M. Kaplan, M. W. Anderson, S. J. Rothfuss, R. Silasi, R. S. Keshari, M. Ferracin, C. Ivan, C. Rodriguez-Aguayo, G. Lopez-Berestein, C. Georgescu, P. P. Banerjee, R. Basar, Z. Li, D. Horst, C. Vasilescu, M. T. S. Bertilaccio, K. Rezvani, F. Lupu, S. C. Yeung, G. A. Calin, Anti-miR-93-5p therapy prolongs sepsis survival by restoring the peripheral immune response, J Clin Invest 133(14) (2023).
[22] F. Benz, S. Roy, C. Trautwein, C. Roderburg, T. Luedde, Circulating MicroRNAs as Biomarkers for Sepsis, Int J Mol Sci 17(1) (2016).
[23] X. Zheng, Y. Zhang, S. Lin, Y. Li, Y. Hua, K. Zhou, Diagnostic significance of microRNAs in sepsis, PLOS One 18(2) (2023) e0279726.
[24] B. Shrum, R. V. Anantha, S. X. Xu, M. Donnelly, S. M. Haeryfar, J. K. McCormick, T. Mele, A robust scoring system to evaluate sepsis severity in an animal model, BMC Res Notes 7 (2014) 233.
[25] Y. S. Tan, Y. L. Lei, Generation and Culture of Mouse Embryonic Fibroblasts, Methods Mol Biol 1960 (2019) 85-91.
[26] J. S. Riley, S. W. Tait, Mitochondrial DNA in inflammation and immunity, EMBO Rep 21(4) (2020) e49799.
[27] O. P. B. Wiklander, M. A. Brennan, J. Lotvall, X. O. Breakefield, S. El Andaloussi, Advances in therapeutic applications of extracellular vesicles, Sci Transl Med 11(492) (2019).
[28] J. C. Lui, W. Chen, K. M. Barnes, J. Baron, Changes in gene expression associated with aging commonly originate during juvenile growth, Mech Ageing Dev 131(10) (2010) 641-9.
[29] C. Harris, S. DiRusso, T. Sullivan, D. L. Benzil, Mortality risk after head injury increases at 30 years, J Am Coll Surg 197(5) (2003) 711-6.
[30] O. Wongweerakit, O. Akaraborworn, B. Sangthong, K. Thongkhao, Age as the Impact on Mortality Rate in Trauma Patients, Crit Care Res Pract 2022 (2022) 2860888.
[31] A. Keren, M. Bertolini, Y. Keren, Y. Ullmann, R. Paus, A. Gilhar, Human organ rejuvenation by VEGF-A: Lessons from the skin, Sci Adv 8(25) (2022) eabm6756.
[32] A. L. Tang, Y. Peng, M. J. Shen, X. Y. Liu, S. Li, M. C. Xiong, N. Gao, T. P. Hu, G. Q. Zhang, Prognostic role of elevated VEGF in sepsis: A systematic review and meta-analysis, Front Physiol 13 (2022) 941257.
[33] A. Wagatsuma, Effect of aging on expression of angiogenesis-related factors in mouse skeletal muscle, Exp Gerontol 41(1) (2006) 49-54.
[34] S. Anand, D. A. Cheresh, MicroRNA-mediated regulation of the angiogenic switch, Curr Opin Hematol 18(3) (2011) 171-6.
[35] T. Wurdinger, B. A. Tannous, O. Saydam, J. Skog, S. Grau, J. Soutschek, R. Weissleder, X. O. Breakefield, A. M. Krichevsky, miR-296 regulates growth factor receptor overexpression in angiogenic endothelial cells, Cancer Cell 14(5) (2008) 382-93.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

We claim:

1. A cosmeceutical or pharmaceutical compositions comprising:
(i) extracellular vesicles isolated from a biological sample obtained from a young donor (Young EVs, or Y-EVs);
(ii) miRNA-296-5p isolated from a biological sample obtained from a young donor subject (Y-miRNA-296-5p) or a functional variant thereof or a vector comprising a nucleic acid encoding miRNA-296-5p isolated from a biological sample obtained from a young donor; and/or
(iii) miR-541-5p isolated from a biological sample obtained from a young donor subject (Y-miR-541-5p) or a functional variant thereof or a vector comprising a nucleic acid encoding miRNA-296-5p isolated from a biological sample obtained from a young donor, optionally, wherein the composition is cell-free.

2. The composition of claim 1, wherein the young donor subject is a human.

3. The composition of claim 1, wherein the young donor is less than 20 years of age.

4. The composition of claim 1, wherein the young donor has an age equivalent to 5-6 week old C57BL/6 mice.

5. The composition of claim 1, comprising mature has-mir-296-5p (AGGGCCCCCCCUCAAUCCUGU (SEQ ID NO:14)) or a functional variant thereof.

6. The composition of claim 5, wherein the functional variant of SEQ ID NO:14 comprises a sequence having at least 70, 75, 80, 85, 90, 95 and up to 99% sequence identity to SEQ ID NO:14.

7. The composition of claim 5, wherein the functional variant is a mimic of SEQ ID NO: 17 or an agomir of SEQ ID NO:14.

9. The composition claim 1, comprising mature hsa-miR-541-5p (AAAGGAUUCUGCUGUCGGUCCCACU (SEQ ID NO:16)); or a functional variant thereof.

10. The composition of claim 5, wherein the functional variant of SEQ ID NO:16 comprises a sequence having at least 70, 75, 80, 85, 90, 95 and up to 99% sequence identity to SEQ ID NO:16.

11. The composition of claim 5, wherein the functional variant is a mimic of SEQ ID NO: 16 or an agomir of SEQ ID NO:16, SEQ ID NO:17 or SEQ ID NO:18.

12. The composition of claim 1, wherein components (i), (ii) and/or (iii) are in an effective amount to reduce intracellular pro-inflammatory cytokines selected from the group consisting of as IL (interleukin)-1β, TNF-α (tumor necrosis factor alpha) and IL-6 and/or inos (inducible nitric oxide synthase), following administration to a subject.

13. A therapeutic or non-therapeutic method of treating a subject, comprising administering to the subject the composition of claim 1.

14. The method of claim of claim 13, wherein composition reduces expression of pro-inflammatory cytokines selected from the group consisting of as IL (interleukin)-1β, TNF-α (tumor necrosis factor alpha) and IL-6 and/or inos (inducible nitric oxide synthase) in cells in of the subject.

15. The method of claim 13, comprising contacting skin of the subject with the pharmaceutical composition, wherein the subject has an inflammatory skin condition or wherein the subject has signs or no signs of skin aging.

16. The method of claim 13, wherein the subject has one or more conditions selected from the group consisting of eczema, psoriasis, acne, seborrheic dermatitis, rosacea, erythema nodosum, dermatitis, poison ivy, poison oak; drug rashes, hidradenitis suppurativa, cutaneous lupus erythematosus, lichen sclerosis, alopecia areata, and vitiligo.

17. The method of claim 13, wherein the subject has been diagnosed with sepsis.

18. The method of claim 13, comprising administering the composition to a topical wound site.
